SEQUENCE LISTING (46 sequences)

SEQ ID NO: 1 - protein, 6 aa, Shigella flexneri
ELDGVD

SEQ ID NO: 2 - protein, 13 aa, Shigella flexneri
SADSHAFSGKTKS

SEQ ID NO: 3 - protein, 3 aa, Shigella flexneri
KDN

SEQ ID NO: 4 - protein, 9 aa, Shigella flexneri
AFGKYNVDN

SEQ ID NO: 5 - protein, 15 aa, Shigella flexneri
SGAGSADGGYSDNYT

SEQ ID NO: 6 - protein, 3 aa, Shigella flexneri
ESG

SEQ ID NO: 7 - protein, 11 aa, Shigella flexneri
GNFASLGTKHY

SEQ ID NO: 8 - protein, 6 aa, Shigella flexneri
YHLTED

SEQ ID NO: 9 - protein, 10 aa, Shigella flexneri
GKTFRWKDGD

SEQ ID NO: 10 - protein, 5 aa, Shigella flexneri
LLNNG

SEQ ID NO: 11 - protein, 13 aa, Shigella flexneri
ASGEKRIKGEKDS

SEQ ID NO: 12 - protein, 13 aa, Yersinia pestis
MTEVLRNSRYRCD

SEQ ID NO: 13 - protein, 13 aa, Yersinia pestis
LSKKMGRTQPTQY

SEQ ID NO: 14 - protein, 13 aa, Yersinia pestis
FGQSPTVFMNGLS

SEQ ID NO: 15 - protein, 13 aa, Yersinia pestis
NGSPDFVAFKSEL

SEQ ID NO: 16 - protein, 13 aa, Yersinia pestis
FKSELIQLIKERG

SEQ ID NO: 17 - protein, 13 aa, Yersinia pestis
FFRIEEGLATSFM

SEQ ID NO: 18 - protein, 13 aa, Yersinia pestis
MNMDLFRNYPPVH

SEQ ID NO: 19 - protein, 13 aa, Yersinia pestis
NSDLLKKEYFDYE

SEQ ID NO: 20 - protein, 13 aa, Yersinia pestis
YDKPEDPLKSSVA

SEQ ID NO: 21 - protein, 13 aa, Yersinia pestis
QKVATRISEYENM

SEQ ID NO: 22 - protein, 13 aa, Yersinia pestis
KEQLIKNAKRIIR

SEQ ID NO: 23 - protein, 13 aa, Yersinia pestis
DGSRADALKRILI

SEQ ID NO: 24 - protein, 13 aa, Yersinia pestis
DALKRILIAPFFF

SEQ ID NO: 25 - protein, 13 aa, Yersinia pestis
TIEGETYKWPDLE

SEQ ID NO: 26 - protein, 13 aa, Yersinia pestis
GIIGSALMSAIKG

SEQ ID NO: 27 - protein, 13 aa, Yersinia pestis
GKDAVNELMKSYW

SEQ ID NO: 28 - protein, 13 aa, Yersinia pestis
VNELMKSYWEPLW

SEQ ID NO: 29 - DNA, 4235 bp, Bacillus anthracis
aagcttctgtcattcgtaaatttcaaatagaacgtaaatttagacttctcatcattaaaaatgaaaaatcttatctttttgattctattgtatatttttattaaggtgtttaatagttagaaaagacagttgatgctattactccagataaaatatagctaaccataaatttattaaagaaaccttgttgttctaaataatgattttgtggattccggaatagatactggtgagttagctctaattttatagtgatttaactaacaatttataaagcagcataattcaaattttttaattgatttttcctgaagcatagtataaaagagtcaaggtcttctagacttgactcttggaatcattaggaattaacaatatatataatgcgctagacagaatcaaattaaatgcaaaaatgaatattttagtaagagatccatatcattatgataataacggtaatattgtaggggttgatgattcatatttaaaaaacgcatataagcaaatacttaattggtcaagcgatggagtttctttaaatctagatgaagatgtaaatcaagcactatctggatatatgcttcaaataaaaaaaccttcaaaccacctaacaaacagcccagttacaattacattagcaggcaaggacagtggtgttggagaattgtatagagtattatcagatggagcaggattcctggatttcaataagtttgatgaaaattggcgatcattagtagatcctggtgatgatgtttatgtgtatgctgttactaaagaagattttaatgcagttactcgagatgaaaatggtaatatagcgaataaattaaaaaacaccttagttttatcgggtaaaataaaagaaataaacataaaaactacaaatattaatatatttgtagtttttatgtttattatatacctcctattttatattattagtagcacagtttttgcaaatcatgtaattgtatacttatctatgtagaggtatcacaacttatgaatagtgtattttattgaacgttggttagcttggacagttgtatggatatgcatactttataacgtataaaatttcacgcaccacaataaaactaatttaacaaaaacaaaaacacacctaagatcattcagttcttttaataaggagctgcccaccaagctaaacctaaataatctttgtttcacataaggtttttttctaaatatacagtgtaagttattgtgaatttaaccagtatatattaaaaatgttttatgttaacaaattaaattgtaaaacccctcttaagcatagttaagaggggtaggttttaaattttttgttgaaattagaaaaaataataaaaaaacaaacctattttctttcaggttgtttttgggttacaaaacaaaaagaaaacatgtttcaaggtacaataattatggttctttagctttctgtaaaacagccttaatagttggatttatgactattaaagttagtatacagcatacacaatctattgaaggatatttataatgcaattccctaaaaatagttttgtataaccagttcttttatccgaactgatacacgtattttagcataatttttaatgtatcttcaaaaacagcttctgtgtccttttctattaaacatataaattcttttttatgttatatatttataaaagttctgtttaaaaagccaaaaataaataattatctctttttatttatattatattgaaactaaagtttattaatttcaatataatataaatttaattttatacaaaaaggagaacgtatatgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtgaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataaggtaattctaggtgatttttaaattatctaaaaaacagtaaaattaaaacatactctttttgtaagaaatacaaggagagtatgttttaaacagtaatctaaatcatcataatcctttgagattgtttgtaggatcc

SEQ ID NO: 30 - protein, 735 aa, Bacillus anthracis
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 31 - DNA, 2396 bp, Bacillus anthracis
ccccttgtacagggggcgggcggtcatggtgatgtaggtatgcacgtaaaagagaaagagaaaaataaagatgagaataagagaaaagatgaagaacgaaataaaacacaggaagagcatttaaaggaaatcatgaaacacattgtaaaaatagaagtaaaaggggaggaagctgttaaaaaagaggcagcagaaaagctacttgagaaagtaccatctgatgttttagagatgtataaagcaattggaggaaagatatatattgtggatggtgatattacaaaacatatatctttagaagcattatctgaagataagaaaaaaataaaagacatttatgggaaagatgctttattacatgaacattatgtatatgcaaaagaaggatatgaacccgtacttgtaatccaatcttcggaagattatgtagaaaatactgaaaaggcactgaacgtttattatgaaataggtaagatattatcaagggatattttaagtaaaattaatcaaccatatcagaaatttttagatgtattaaataccattaaaaatgcatctgattcagatggacaagatcttttatttactaatcagcttaaggaacatcccacagacttttctgtagaattcttggaacaaaatagcaatgaggtacaagaagtatttgcgaaagcttttgcatattatatcgagccacagcatcgtgatgttttacagctttatgcaccggaagcttttaattacatggataaatttaacgaacaagaaataaatctatccttggaagaacttaaagatcaacggatgctggcaagatatgaaaaatgggaaaagataaaacagcactatcaacactggagcgattctttatctgaagaaggaagaggacttttaaaaaagctgcagattcctattgagccaaagaaagatgacataattcattctttatctcaagaagaaaaagagcttctaaaaagaatacaaattgatagtagtgattttttatctactgaggaaaaagagtttttaaaaaagctacaaattgatattcgtgattctttatctgaagaagaaaaagagcttttaaatagaatacaggtggatagtagtaatcctttatctgaaaaagaaaaagagtttttaaaaaagctgaaacttgatattcaaccatatgatattaatcaaaggttgcaagatacaggagggttaattgatagtccgtcaattaatcttgatgtaagaaagcagtataaaagggatattcaaaatattgatgctttattacatcaatccattggaagtaccttgtacaataaaatttatttgtatgaaaatatgaatatcaataaccttacagcaaccctaggtgcggatttagttgattccactgataatactaaaattaatagaggtattttcaatgaattcaaaaaaaatttcaaatatagtatttctagtaactatatgattgttgatataaatgaaaggcctgcattagataatgagcgtttgaaatggagaatccaattatcaccagatactcgagcaggatatttagaaaatggaaagcttatattacaaagaaacatcggtctggaaataaaggatgtacaaataattaagcaatccgaaaaagaatatataaggattgatgcgaaagtagtgccaaagagtaaaatagatacaaaaattcaagaagcacagttaaatataaatcaggaatggaataaagcattagggttaccaaaatatacaaagcttattacattcaacgtgcataatagatatgcatccaatattgtagaaagtgcttatttaatattgaatgaatggaaaaataatattcaaagtgatcttataaaaaaggtaacaaattacttagttgatggtaatggaagatttgtttttaccgatattactctccctaatatagctgaacaatatacacatcaagatgagatatatgagcaagttcattcaaaagggttatatgttccagaatcccgttctatattactccatggaccttcaaaaggtgtagaattaaggaatgatagtgagggttttatacacgaatttggacatgctgtggatgattatgctggatatctattagataagaaccaatctgatttagttacaaattctaaaaaattcattgatatttttaaggaagaagggagtaatttaacttcgtatgggagaacaaatgaagcggaattttttgcagaagcctttaggttaatgcattctacggaccatgctgaacgtttaaaagttcaaaaaaatgctccgaaaactttccaatttattaacgatcagattaagttcattattaactcataagtaatgtattaaaaattttcaaatggatttaataataataataataataa

SEQ ID NO: 32 - protein, 776 aa, Homo sapiens
AGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 33 - DNA, 558 bp, Hepatitis B virus
atggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatgtcgggaatctcaatgttag

SEQ ID NO: 34 - protein, 149 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVV

SEQ ID NO: 35 - DNA, 756 bp, Artificial sequence
  Polynucleotide encoding chimeric immunogenic peptide
atggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatcttggtggtggtggggctgatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaggtggtggtggtgcctccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgtt

SEQ ID NO: 36 - protein, 252 aa, Artificial sequence
  Immunogenic peptide
MDIDPYKEFGATVELLSFLPSDFFPSVRDLLDTASALYREALESPEHCSPHHTALRQAILCWGELMTLATWVGNNLGGGGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKEGGGGASRDLVVNYVNTNMGLKIRQLLWFHISCLTFGRETVLEYLVSFGVWIRTPPAYRPPNAPILSTLPETTVV

SEQ ID NO: 37 - protein, 4 aa, Artificial sequence
  Linker peptide
GGGG

SEQ ID NO: 38 - protein, 25 aa, Yersinia pestis
NHTKIMASDGTEALVGGHNMNMDLF

SEQ ID NO: 39 - protein, 25 aa, Homo sapiens
NHEKLVIIDQSVAFVGGINLAYGRW

SEQ ID NO: 40 - protein, 26 aa, Yersinia pestis
GNHAKLMIIDDELYVVGSDNLYPGYL

SEQ ID NO: 41 - protein, 26 aa, Homo sapiens
YVHSKLLIADDNTVIIGSANINDRSM

SEQ ID NO: 42 - protein, 5 aa, Artificial sequence
  Linker peptide
GGGGG

SEQ ID NO: 43 - protein, 6 aa, Artificial sequence
  Linker peptide
GGGGGG

SEQ ID NO: 44 - protein, 5 aa, Artificial sequence
  Linker peptide
GSAGS

SEQ ID NO: 45 - protein, 6 aa, Artificial sequence
  Linker peptide
GSAGSA

SEQ ID NO: 46 - protein, 7 aa, Artificial sequence
  Linker peptide
GSAGSAG

(19) United States
(12) Patent Application Publication
(10) Pub. No.: US 2003/0235818 A1
(43) Pub. Date: Dec. 25, 2003

(54) Immunogenic peptides, and method of identifying same

(76) Inventors: Vsevolod Katritch, San Diego, CA (US); Andrew Bordner, San Diego, CA (US); Robert Deans, Claremont, CA (US); Mary Sumner, San Diego, CA (US)

Correspondence Address: LISA A. HAILE, J.D., PH.D., GRAY CARY WARE & FREIDENRICH LLP, Suite 1100, 4365 Executive Drive, San Diego, CA, 92121-2133, US

(21) Appl. No.: 10/410,647

(22) Filed: Apr. 8, 2003

Related U.S. Application Data
(60) Provisional application No. 60/371,250, filed on Apr. 8, 2002.
(60) Provisional application No. 60/371,256, filed on Apr. 8, 2002.
(60) Provisional application No. 60/373,668, filed on Apr. 17, 2002.

(51) Int. Cl. C12Q 1/70; G06F 19/00; G01N 33/48; G01N 33/50

(52) U.S. Cl. 435/5; 702/19

(57) ABSTRACT

Immunogenic peptides, polynucleotides encoding immunogenic peptides, antibodies that selectively bind immunogenic peptides and methods of identifying immunogenic peptides are provided. The immunogenic peptides are representative of a structural element of a target protein. The methods of the invention are useful for identifying immunogenic peptides of a target protein having a known three dimensional structure, or of a target protein having a known amino acid sequence but an unknown three dimensional structure.

### CROSS REFERENCE TO RELATED APPLICATION(S)

This application claims the benefit of priority under 35 U.S.C. §119(e) of U.S. Serial No. 60/371,250, filed Apr. 8, 2002; U.S. Serial No. 60/371,256, filed Apr. 8, 2002; and U.S. Serial No. 60/373,668, filed Apr. 17, 2002, the entire content of each of which is incorporated herein by reference.

### GRANT INFORMATION

This invention was made in part with government support under Grants Nos. 1-R43-AI52969-01 and 1-R43-C₁₀₀₀₈₆-01-A1 awarded by the National Institutes of Health. The government has certain rights in this invention.

### BACKGROUND OF THE INVENTION

1. Field of the Invention

The invention relates generally to methods of identifying epitopes useful as immunogens, and more specifically to methods of identifying immunogenic peptides representative of a structural element of a target protein, to immunogenic peptides identified using such a method, and to methods of using an immunogenic peptide to stimulate an immune response in a subject.

2. Background Information

Infection is a leading cause of morbidity and mortality in the human population. One of the greatest achievements of modem medicine has come from the development of vaccines, which have eradicated or virtually eliminated several human diseases. Vaccination is the single most successful manipulation of the immune system so far, because it takes advantage of the immune system's natural specificity and inducibility. Nevertheless, there are many important infectious diseases for which there is still no effective vaccine. Some vaccines are based on attenuated live microorganisms, but these carry some risk and are potentially lethal to immunosuppressed or immunodeficient individuals. Better techniques for developing live-attenuated vaccines, or vaccines that incorporate both immunogenic components and protective antigens of pathogens, are therefore being sought. Some current viral vaccines are based on live attenuated virus, but many bacterial vaccines are based on components of the micro-organism, including components of the toxins that it produces. The effectiveness of these types of vaccines is limited because of the difficulty in completely separating toxicological effects from a component's ability to induce specific immune protection. Developing a protective response to carbohydrate antigens is one approach and can be enhanced by conjugation to a protein.

One promising approach comes from vaccines based on peptide epitopes, or portions of peptides of a microorganism that are able to induce an immune response in the infected subject. Vaccines based on peptide epitopes are still at an experimental stage and have the problem that peptide portions removed from a protein do not necessarily maintain native conformation once removed from the protein, and may be only very weakly immunogenic. Thus a need exists for improved methods of identifying or characterizing immunogenic peptide epitopes useful for vaccines.

### SUMMARY OF THE INVENTION

The present invention relates to methods of identifying immunogenic peptides, which are representative of a structural element of a target protein. The methods of the invention utilize molecular modeling to identify epitopes that can be useful in preparing an immunogenic peptide and/or to confirm that a selected epitope, when linked to a scaffold protein, has a three dimensional conformation corresponding to the structure of the epitope in the target protein from which it was derived. The methods of the invention are useful for identifying immunogenic peptides of a target protein having a known three dimensional structure, or of a target protein having a known amino acid sequence but unknown three dimensional structure.

Accordingly, in one embodiment, the present invention relates to a method for identifying an immunogenic peptide representative of a structural element of a target protein having a known three dimensional structure. Such a method can be performed, for example, by selecting epitopes of the target protein based on the three dimensional structure of the target protein, thereby obtaining selected epitopes; detecting, in a molecular model of a polypeptide comprising a selected epitope linked to a scaffold protein, an epitope having a three dimensional conformation corresponding to the three dimensional structure of the epitope in the target protein, thereby identifying a candidate immunogenic peptide representative of a structural element of the target protein; and detecting that antibodies induced by the candidate immunogenic peptide selectively bind to the target protein, thereby identifying an immunogenic peptide representative of a structural element of the target protein. The invention also provides an immunogenic peptide, or a plurality thereof, identified according to such a method.

Selected epitopes can include, for example, peptide portions of the target protein that are on a surface of the target protein, including peptide portions of the target protein that can be bound by an antibody in the native protein; and/or peptide portions of the target protein that are likely to be soluble in an aqueous solution. The scaffold protein, to which an epitope can be linked, can be any polypeptide that provides a defined and stable three dimensional conformation of the epitope linked thereto. As such, the scaffold protein can be a synthetic poly(amino acid), which can be a homopolymer or heteropolymer, for example, a homopolymer such as poly(lysine), or can be a naturally occurring protein, or peptide portion thereof, for example, a viral coat protein such as a Hepatitis B core protein.

An advantage of using a scaffold protein such as a viral coat protein, wherein a plurality of immunogenic peptide comprising the scaffold protein can be assembled to form a complex, is that a plurality of such scaffold proteins, each of which contains a selected epitope (i.e., the plurality of immunogenic peptides), can be assembled to form a virus-like particle, thus presenting a clustered plurality of the epitopes. A further advantage of assembling a plurality of immunogenic peptides is that the epitopes of the plurality of immunogenic peptides can all be the same, can include some epitopes that are the same and some that are different. Where the immunogenic peptides of a plurality include epitopes that are different, the epitopes can be different epitopes of the same target protein, or can e epitopes of different target proteins.

A selected epitope can be linked directly to the scaffold protein, or can be linked via a linker moiety. An advantage of linking the selected epitope to the scaffold protein via a linker moiety is that the size of the linker moiety can be varied, thus providing a means to obtain a variety polypeptides, each of which include a selected epitope linked to a scaffold protein, in which the selected epitope is in different conformations and, therefore, an opportunity to identify a polypeptide in which the selected epitope is in a conformation corresponding to the conformation of the epitope in the native target protein. In one embodiment, the linker moiety is a peptide such that the polypeptide containing the linker moiety, selected epitope, and scaffold protein is a fusion protein, which can be prepared using chemical peptide synthesis methods or can be expressed from a polynucleotide encoding the polypeptide.

The target protein, from which epitopes are derived, can be any protein for which it is desired to identify an immunogenic peptide. In one aspect, the target protein is a protein of an infectious microorganism, for example, a cell surface protein, a toxin, a protein involved in infectiousness or spread of the microorganism, or any other protein of the microorganism, particularly a protein that, in a living subject or a sample obtained from a living subject, can be contacted with an antibody. The infectious microorganism can be a eukaryotic or prokaryotic microorganism, including, for example, a bacterium, a protozoan, a yeast, or a fungus. For example, the infectious microorganism can be a bacterium that causes anthrax (e.g., Bacillus anthracis), in which case the target protein can be an anthrax protective antigen (SEQ ID NO:30) or an anthrax lethal factor (SEQ ID NO:32) or both.

The present invention also relates to an isolated peptide of a B. anthracis protective antigen. Such isolated peptides of the invention are exemplified by a peptide consisting of amino acid residues 606 to 705 of SEQ ID NO:30; amino acid residues 606 to 735 of SEQ ID NO:30 amino acid residues 606 to 706 of SEQ ID NO:30; amino acid residues 606 to 704 of SEQ ID NO:30; amino acid residues 606 to 734 of SEQ ID NO:30; amino acid residues 607 to 703 of SEQ ID NO:30; amino acid residues 606 to 732 of SEQ ID NO:30; amino acid residues 604 to 707 of SEQ ID NO:30; amino acid residues 606 to 730 of SEQ ID NO:30; or amino acid residues 606 to 733 of SEQ ID NO:30. The invention also provides an isolated antibody that selectively binds such a peptide, provided the antibody does not substantially bind a peptide comprising amino acid residues 596 to 735 of SEQ ID NO:30; amino acid residues 679 to 693 of SEQ ID NO:3; amino acid residues 703 to 722 of SEQ ID NO:30, or amino acid residues 671 to 721 of SEQ ID NO:30. The invention also provides an isolated polynucleotide encoding a peptide as recited above. In addition, the invention provides an isolated peptide consisting of amino acid residues 17 to 153 of SEQ ID NO:30; amino acid residues 261 to 454 of SEQ ID NO:30; or amino acid residues 487 to 594 of SEQ ID NO:30, as well as polynucleotides encoding such peptides, and an antibody that specifically bind such a peptide, provided the antibody does not substantially bind to a polypeptide comprising SEQ ID NO:30.

The present invention also relates to a composition, which includes at least a first epitope of a target protein, for example, a peptide of the invention, operatively linked to at least a first heterologous molecule. Thus, in one embodiment, the composition includes at least a first peptide of the invention operatively linked to at least a first heterologous molecule. For example, the first peptide can be a peptide consisting of amino acid residues 606 to 705 of SEQ ID NO:30; amino acid residues 606 to 735 of SEQ ID NO:30; amino acid residues 606 to 706 of SEQ ID NO:30; amino acid residues 606 to 704 of SEQ ID NO:30; amino acid residues 606 to 734 of SEQ ID NO:30; amino acid residues 607 to 703 of SEQ ID NO:30; amino acid residues 606 to 732 of SEQ ID NO:30; amino acid residues 604 to 707 of SEQ ID NO:30; amino acid residues 606 to 730 of SEQ ID NO:30; or amino acid residues 606 to 733 of SEQ ID NO:30; wherein one or more of such peptides, which can be the same or different, is operatively linked to at least a first heterologous molecule.

The heterologous molecule, which is operatively linked to the peptide, can be any molecule, including, for example, a heterologous peptide, a peptidomimetic, a polynucleotide, a small organic molecule, or a combination of such molecules. In one aspect, at least a first heterologous molecule is a linker moiety, for example, a peptide linker such as an oligo(amino acid) (e.g., an oligo(glycine) linker or a (glycine-serine-alanine)_{N} linker. In another aspect, at least a first heterologous peptide, which is operatively linked to an epitope, particularly a peptide of the invention, is a scaffold protein, which provides conformational stability to the peptide. The scaffold protein can be a naturally occurring protein, or a peptide portion thereof, for example, a viral coat protein such as a Hepatitis B core (HBc) protein, or a peptide portion thereof such as a first peptide portion set forth as amino acid residues 1 to 75, 1 to 76, 1 to 77, or 1 to 78 of SEQ ID NO:34 and a second peptide portion set forth as amino acid residues 79 to 149, 80 to 149, 81 to 149, or 82 to 149 of SEQ ID NO:34, wherein the epitope/peptide is operatively linked at one terminus, particularly the N-terminus, to the C-terminal amino acid of the first portion of SEQ ID NO:34, e.g., to amino acid residue 75, 76, 77, or 78 of SEQ ID NO:34, and at the other terminus, particularly the C-terminus, to the N-terminal amino acid of the second portion of SEQ ID NO:34, e.g., to amino acid residue 79, 80, 81, or 82 of SEQ ID NO:34.

In still another aspect, the composition comprises an epitope or a peptide of the invention operatively linked to at least a first linker molecule, for example, a first and second linker molecule positioned at each terminus of the epitope/peptide, and further operatively linked, via the linker molecule(s), to a scaffold protein. For example, where the scaffold protein comprises two peptide portions of a viral coat protein such as amino acid residues 1 to 77 of SEQ ID NO:34 and amino acid residues 80 to 149 of SEQ ID NO:34, the epitope/peptide can be operatively linked to amino acid residue 77 via a first linker moiety and operatively linked to amino acid residue via a second linker moiety, wherein the first linker moiety and the second linker moiety are the same or different. In yet another aspect, the components of a composition of the invention are peptides, and the composition comprises a fusion protein, which, conveniently, can be encoded by and expressed from a polynucleotide.

A composition of the invention can further include at least a second heterologous molecule (e.g., a linker and a scaffold protein); a second and at least a third heterologous molecule (e.g., a first linker, a second, linker, and a scaffold protein); a second, a third, and at least a fourth heterologous molecule (e.g., a first linker, a second linker, a first portion of a scaffold protein, and a second portion of a scaffold protein), which can be in operative linkage. As such, in one aspect, a composition of the invention comprises an immunogenic peptide representative of a structural element of a target protein. For example, the composition can include, in operative linkage, a first portion of a scaffold protein, a first peptide linker, a peptide of the invention, a second peptide linker, and a second portion of the scaffold protein (e.g., from N-terminus to C-terminus). Such a composition is exemplified herein by the immunogenic composition having an amino acid sequence as set forth in SEQ ID NO:36, which includes, from N-terminus to C-terminus, a first portion of a scaffold protein (amino acid residues 1 to 77 of SEQ ID NO:34, a first linker moiety (triglycine), a peptide of the invention (amino acid residues 606 to 704 of SEQ ID NO:30), a second linker moiety (triglycine), and a second portion of a scaffold protein (amino acid residues 80 to 149 of SEQ ID NO:34).

In another aspect, the first (or other) heterologous molecule of a composition of the invention comprises a tag operatively linked to a peptide of the invention. The tag can be any type of tag useful for detecting the presence of the peptide of the invention, for isolating the peptide, or the like. For example, the tag can be a peptide tag such as oligo(histidine) tag (e.g., 6×His), or can be a small organic molecule such as biotin, or the like. The tag further can be a detectable label, for example, a fluorescent moiety, a luminescent moiety, or a chemiluminescent moiety.

In another embodiment, a composition of the invention comprises a plurality of polypeptides, each polypeptide of the plurality comprising an immunogenic peptide representative of a structural element of a target protein, particularly a polypeptide comprising a peptide of the invention operatively linked to a viral coat protein. In one aspect of such a composition, the polypeptides of the plurality are assembled to form a virus-like particle.

A composition of the invention can be present in a solid form, for example, in a lyophilized form or a crystallized form, or can be in a solution, which can be an aqueous or non-aqueous solution, depending on the physico-chemical characteristics of the composition. In addition, the composition can be formulated for administration to a subject, for example, to stimulate antibodies against a target protein such as an anthrax protein. As such, the composition can further include other reagents useful for stimulating the immune response, for example, an adjuvant, or otherwise beneficial to the subject receiving the composition, for example, a therapeutic agent or a nutrient.

Where a composition of the invention is a fusion protein, the present invention further relates to a polynucleotide encoding the composition. A polynucleotide of the invention is exemplified by SEQ ID NO:35, which encodes a composition comprising a first portion of a scaffold protein (amino acid residues 1 to 77 of SEQ ID NO:34), a first linker moiety (triglycine), a peptide of the invention (amino acid residues 606 to 704 of SEQ ID NO:30), a second linker moiety (triglycine), and a second portion of a scaffold protein (amino acid residues 80 to 149 of SEQ ID NO:34). The polynucleotide can be contained in a vector, which can be a cloning vector, thus providing a means to prepare a desired amount of the polynucleotide encoding the composition or to manipulate the polynucleotide; or can be an expression vector, thus providing a means to produce the encoded fusion protein. For example, the vector can be a viral vector, particularly a viral vector derived from a virus having a desired host or cell type specificity (e.g., a retrovirus, an adenovirus, a herpesvirus, or an adeno-associated virus). The polynucleotide, which can, but need not be contained in a vector, also can be contained in a matrix that facilitates manipulation of the polynucleotide or introduction of the polynucleotide into a cell, for example, a matrix useful for forming liposomes or microbubbles.

The present invention also relates to method of stimulating an immune response in a subject. Such a method can be performed, for example, by administering a composition of the invention, particularly an immunogenic peptide representative of a structural element of a target protein, or a polynucleotide encoding the composition, to a subject under conditions suitable for stimulating an immune response. Such conditions will depend, for example, on the material being administered. Where an immunogenic peptide is administered, it can be administered, for example, sub-dermally or mucosally, and can, but need not be formulated with an adjuvant, and can be administered as an initial dose and in booster doses, either or any of which can include an adjuvant. Where a polynucleotide encoding an immunogenic peptide is administered, the polynucleotide is administered such that it can enter cells in the subject and the encoded immunogenic peptide can be expressed. In one aspect, the encoded immunogenic can further include a peptide sequence that directs translocation of the immunogenic to the cell surface and/or secretion of the immunogenic peptide from the cell such that it can stimulate an immune response in the subject.

A subject treated according to a method of the invention can be any subject in which it is desired to stimulate an immune response using an immunogenic peptide of the invention. As such, the subject can be a vertebrate subject, including, for example, a mammalian subject such as a rabbit, goat, mouse, or other mammal, thus providing a means to generate antibodies specific for the target protein for which the immunogenic peptide represents a structural element. Accordingly, the method can further include isolating such antibodies from the mammalian subject. In one embodiment, the subject treated according to a method of the invention is a human subject, wherein the immune response in the subject can be stimulated to protect the subject from harm due to an infectious microorganism that expresses the target protein, or to ameliorate such harm following infection with the microorganism. In one aspect, the subject is a human subject exposed to or at risk of exposure to B. anthracis, and administration of the immunogenic peptide generates a protective immune response in the subject against the signs and symptoms of anthrax.

In another embodiment, a method of the invention is used to identify an immunogenic peptide representative of a structural element of a target protein having a known amino acid sequence but an unknown three dimensional structure. Such a method can be performed, for example, by generating a molecular model of a three dimensional structure of the target protein based on the three dimensional structure of a homologous protein; selecting epitopes of the target protein based on the molecular model of the three dimensional structure of the target protein, thereby obtaining selected epitopes; detecting, in a molecular model of a chimeric polypeptide comprising at least one selected epitope having a constrained structure, an epitope having a three dimensional conformation corresponding to the molecular model of the three dimensional structure of the epitope in the target protein, thereby identifying a candidate immunogenic peptide representative of a structural element of the target protein; and detecting that antibodies induced by the candidate immunogenic peptide selectively bind to the target protein, thereby identifying an immunogenic peptide representative of a structural element of the target protein. Accordingly, the invention provides an immunogenic peptide identified according to such a method. In one aspect, the chimeric polypeptide and, therefore, an immunogenic peptide identified therefrom, is a fusion protein.

According to one aspect of a method of the invention, the chimeric polypeptide, which can be a fusion protein, includes a plurality of linked selected epitopes, wherein the selected epitopes of the plurality are the same, are different, or include some that are the same and others that are different. The chimeric polypeptide can further include at least one linker peptide, which is linked to at least one selected epitope of the chimeric polypeptide. The constrained structure of the selected epitope can be due, for example, to at least one disulfide bond in the chimeric polypeptide, to cyclization of the chimeric polypeptide (e.g., via a peptide bond formed between the C-terminus and N-terminus of the chimeric polypeptide or the selected epitope portion thereof; or via a bond formed between two reactive side chains of amino acid residues in the chimeric polypeptide); or due to linkage of the selected peptide to a scaffold protein, for example, a viral coat protein such as Hepatitis B core protein, or a poly(amino acid) such as poly(lysine). In another aspect of a method of the invention, the selected epitope is linked to a scaffold protein via a linker moiety, for example, a peptide linker.

A target protein, for which an immunogenic peptide is identified according to a method of the invention, can be a protein of an infectious microorganism, particularly a protein involved in infectiveness of microorganism or a deleterious effect caused by the microorganism. The infectious microorganism can be, for example, a protozoan such as Entamoeba histolytica, a yeast such as Candida albicans, or a bacterium such as Shigella flexneri, Yersinia pestis, or Serratia marcescens As such, a target protein can be, for example, an autotransporter of a bacterium such as an autotransporter as set forth in Table 1 (e.g., a Shigella autotransporter such as a Shigella flexneri protease involved in colonization (pic) gene product; or can be, for example, a bacterial protein having an enzymatic activity such as a phospholipase activity, e.g., the activity of a Yersinia murine toxin as expressed by a Yersinia species such as Yersinia pestis.

A method of the invention can further include assembling a plurality of immunogenic peptides, in which the selected epitope is operatively linked to a viral coat protein, into a virus-like particle. The selected epitopes of the immunogenic peptides in such as assembled virus-like particle can be the same or different or a combination of some that are the same and others that are different.

The present invention also relates to an isolated peptide, consisting of a peptide as set forth in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO: 11 (see Table 5), each of which is a peptide of a Shigella flexneri pic gene product. In addition, the present invention relates to an isolated peptide, consisting of a peptide as set forth SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, or SEQ ID NO:28 (see Table 6), each of which is a peptide of a Yersinia pestis murine toxin. Also provided is a polynucleotide encoding a peptide consisting of any of SEQ ID NOS:1 to 28, wherein the polynucleotide can, but need not, be contained in a vector (e.g., a viral vector) and/or in a matrix (e.g. liposomes or microbubbles).

The present invention further relates to a composition, which includes at least a first peptide of the invention operatively linked to at least a first heterologous molecule. In one embodiment, the composition includes a plurality of peptides of the invention, wherein the peptides of the plurality are linked to each other, for example, a plurality of two, three, four, five, six, etc., peptides, which can be the same or different or a combination thereof. In one aspect, the first heterologous molecule can be a linker moiety, for example, a peptide linker or biotin, or can be any heterologous molecule, including any heterologous peptide. In another aspect, the first heterologous peptide comprises a scaffold protein, for example, a poly(amino acid) such as poly(lysine), or a viral coat protein such as a HBc protein (e.g., a first portion of the HBc protein consisting of amino acid residues 1 to 77 of SEQ ID NO:34 and a second portion of the HBc protein consisting of amino acid residues 80 to 149 of SEQ ID NO:34, wherein the peptide of the invention is operatively linked to amino acid residue 77 and to amino acid 80 of SEQ ID NO:34. In another embodiment, the composition includes a plurality of peptides of the invention, wherein each peptide of the plurality is operatively linked to a viral coat protein. In one aspect of this embodiment, the plurality of peptides is assembled, via the viral coat protein component, to form a virus-like particle.

A composition of the invention can first include a second heterologous molecule, a third heterologous molecule, a fourth heterologous molecule, etc. In one aspect, the composition includes at least a second heterologous molecule, and the first heterologous molecule is a linker moiety or a scaffold protein or a portion thereof. For example, composition can include a first heterologous molecule that is a linker moiety and a second heterologous molecule that is a scaffold protein (or a portion thereof), and the linker moiety can operatively link the peptide of the invention (or other selected epitope) to the scaffold protein. In another example, the composition can include, in operative linkage, a first portion of a scaffold protein, a first peptide linker, the peptide of the invention (or other selected epitope), a second peptide linker, and a second portion of the scaffold protein.

The first (or other) heterologous molecule in a composition of the invention also can be a tag, such as a peptide or other small molecule tag, which can, but need not, be a detectable label or provide a means for identifying and/or isolating the composition. The first (or other) heterologous molecule also can be any other molecule of interest, including, for example, a carrier protein; a peptide that directs transport of the composition to an intracellular compartment or to a cell surface, or directs secretion of the composition out of a cell.

In one embodiment, a composition of the invention is an immunogenic composition, which comprises an immunogenic peptide representative of a structural element of a target protein. As such, the composition can be formulated for administration to a subject, for example, by including an adjuvant or other agent that may be desirable for stimulating an immune response. Accordingly, the invention further relates to a method of stimulating an immune response in a subject by administering such an immunogenic composition, or, where the immunogenic composition comprises a polypeptide, a polynucleotide encoding the composition, to a subject under conditions suitable for stimulating an immune response. The immune response stimulated in the subject can be a protective immune response, and the subject can be any subject in need of such an immune response, for example, a mammalian subject such as a human subject.

### BRIEF DESCRIPTION OF THE DRAWINGS

FIG. 1 shows ELISA data where New Zealand White rabbits were immunized intramuscularly with a PA-VLP protein, without adjuvant, and boosted with protein, without adjuvant. Antisera was tested by ELISA for reactivity to PA-VLP.

FIGS. 2A and 2B show the results of ELISA experiments using antisera obtained from rabbits immunized with PA-VLP and cross-reacted with anthrax rPA.

FIG. 3 shows ELISA data illustrating reactivity of specific peptides, “peptide2” (SEQ ID NO:11) and peptide3 (SEQ ID NO:7) to anti-Shigella flexneri antisera.

FIG. 4 shows rabbit antibodies raised against the YMT synthetic peptides recognized the target epitope as a structure on whole YMT protein, when tested by ELISA.

### DETAILED DESCRIPTION OF THE INVENTION

The present invention relates to methods of identifying immunogenic peptides, which are representative of a structural element of a target protein. The methods of the invention utilize molecular modeling to identify epitopes that can be useful in preparing an immunogenic peptide and/or to confirm that a selected epitope, when linked to a scaffold protein, has a three dimensional conformation corresponding to the structure of the epitope in the target protein from which it was derived. The methods of the invention are useful for identifying immunogenic peptides of a target protein having a known three dimensional structure, or of a target protein having a known amino acid sequence but unknown three dimensional structure.

The target protein, from which epitopes are derived, can be any protein for which it is desired to identify an immunogenic peptide. As used herein, the term “immunogenic peptide” means a peptide, identified according to the methods of the present invention, capable of provoking or eliciting an immune response. A target protein is exemplified herein by a protein of an infectious microorganism, for example, a cell surface protein, a toxin, a protein involved in infectiousness or spread of the microorganism, or any other protein of the microorganism, particularly a protein that, in a living subject or a sample obtained from a living subject, can be contacted with an antibody. The infectious microorganism can be a eukaryotic or prokaryotic microorganism, including, for example, a bacterium, a protozoan, a yeast, or a fungus. The infectious microorganism can be, for example, a protozoan such as Entamoeba histolytica, a yeast such as Candida albicans, or a bacterium such as Shigella flexneri, Yersinia pestis, or Serratia marcescens. According to another example, the infectious microorganism can be a bacterium that causes anthrax (e.g., Bacillus anthracis).

The target peptides of the invention can be characterized into two general categories, including those target peptides having a known three dimensional structure, and those target peptides having a known amino acid sequence but an unknown three dimensional structure. In some instances, the three dimensional structure of a target protein is known and can be obtained by searching, for example, a protein database or database of protein structures (see, for example the Protein Data Bank (PDB); see, also, the world wide web, at the url “rcsb.org/pdb/”). A three dimensional structure of a target protein also can be obtained according to methods routinely used in the art for obtaining protein structural information, such as by crystallographic analysis or nuclear magnetic resonance analysis (see, for example, Dunbrack et al., “Meeting review: the Second meeting on the Critical Assessment of Techniques for Protein Structure Prediction (CASP2) (Asilomar, Calif., Dec. 13-16, 1996). Fold Des. 2(2): R27-42, (1997); Fischer and Eisenberg, Protein Sci. 5:947-55, 1996; (see, also, U.S. Pat. No. 5,436,850); Havel, Prog. Biophys. Mol. Biol. 56:43-78, 1991; Lichtarge et al., J. Mol. Biol. 274:325-37, 1997; Matsumoto et al., J. Biol. Chem. 270:19524-31, 1995; SalI et al., J. Biol. Chem. 268:9023-34, 1993; SalI, Molec. Med. Today 1:270-7, 1995a; SalI, Curr. Opin. Biotechnol. 6:437-51, 1995b; SalI et al., Proteins 23: 318-26, 1995c; SalI, Nature Struct. Biol. 5:1029-1032, 1998; U.S. Pat. No. 5,933,819; U.S. Pat. No. 5,265,030, each of which is incorporated herein by reference).

In one embodiment, a method for identifying an immunogenic peptide representative of a structural element of a target protein having a known three dimensional structure is provided. Such a method can be performed, for example, by selecting epitopes of the target protein based on the three dimensional structure of the target protein, thereby obtaining selected epitopes; detecting, in a molecular model of a polypeptide comprising a selected epitope linked to a scaffold protein, an epitope having a three dimensional conformation corresponding to the three dimensional structure of the epitope in the target protein, thereby identifying a candidate immunogenic peptide representative of a structural element of the target protein; and detecting that antibodies induced by the candidate immunogenic peptide selectively bind to the target protein, thereby identifying an immunogenic peptide representative of a structural element of the target protein. The invention also provides an immunogenic peptide, or a plurality thereof, identified according to such a method.

The term “epitope” is used herein as it is generally used in the art to refer to an antigenic determinant, or the structure of an antigen molecule (e.g., a haptenic portion) that interacts with the combining site of an antibody or T cell receptor as a result of molecular complementarity. In general, protein epitopes recognized by antibodies may be continuous or discontinuous depending on whether the amino acid residues forming the epitope are in continuous peptide linkage or are in spatial proximity to each other as a consequence of the tertiary or quaternary structure of the molecule. Such epitopes are typically expressed on the surface of the proteins. Protein epitopes recognized by T cell receptors are peptides generated by enzymatic degradation of the protein molecule and presented on the cell surface in association with class I or class II MHC molecules.

The term “selected epitopes”, as used herein, refers more specifically to epitopes of the target protein that have been selected, based on the three dimensional structure of the target protein as being positioned on the target protein such that they are particularly available to contact with an antibody. As such, selected epitopes can include, for example, peptide portions of the target protein that are on a surface of the target protein, including peptide portions of the target protein that can be bound by an antibody in the native protein; and/or synthetic peptides based on discontinuous peptide portions of a target protein that are in special proximity in the protein; and/or peptide portions of the target protein that are predicted to be soluble in an aqueous solution.

A method of the present invention also includes identifying a candidate immunogenic peptide representative of a structural element of the target protein. An immunogenic peptide is identified by first linking a selected epitope to a molecule that provides a means to maintain the three dimensional structure of a selected epitope, such as a scaffold protein. Once a selected epitope is linked to a scaffold protein, the three dimensional structure of the peptide including both the selected epitope and scaffold protein can be examined, such as according to the molecular modeling systems described below, and compared to the three dimensional structure of the selected epitope naturally contained in a target protein.

Thus, a “candidate immunogenic peptide” is a polypeptide including a selected epitope linked to a protein scaffold, wherein the epitope has a three dimensional conformation corresponding to the three dimensional conformation of the epitope in a target protein. The three dimensional conformation of the epitope in a target protein refers to the conformation of the epitope in a native or naturally-occurring target protein, or the target protein as it can be found in nature as distinct from being artificially produced by man. For example, a target protein that is present in an organism (including viruses, bacteria, protozoa, insects, plants or mammalian tissue, etc) that can be isolated from a source in nature and which has not been intentionally modified by man in the laboratory is native or naturally-occurring.

Molecular modeling systems useful for practicing the methods of the invention can be based on structural information obtained, for example, by searching a protein database or based on structural information determined, for example, by crystallographic analysis or nuclear magnetic resonance analysis or any other method known in the art. Crystal structure coordinates of a target protein can be used to detect a polypeptide comprising a selected epitope linked to a scaffold protein corresponding to the three dimensional structure of the epitope in the target protein.

Computer programs for carrying out the activities necessary to detect candidate immunogenic peptides are well known in the art. For example, methodology for epitope modeling can be implemented in the Internal Coordinates Modeling™ (ICM™) software suite from Molsoft, LLC (see, for example, the world wide web at URL “molsoft.com”). The ICM™ software suite relies on internal coordinate definition of the molecular object combined with computationally efficient Biased Probability Monte Carlo (BPMC; ref 1; citations for references identified by “ref” follow Example 3, below), an extended force field including surface terms, electrostatics with the boundary element solution of the Poisson equation (ref 2), side chain entropy terms, and a fast algorithm for calculating molecular surfaces (ref 3). The ICM™ software suite algorithm has demonstrated accurate loop predictions in several protein engineering applications (refs 4, 5, 6, 7) and is extensively used as a part of homology modeling algorithms (refs 8, 9).

The scaffold protein, to which an epitope can be linked, can be any polypeptide that provides a defined and stable three dimensional conformation of the epitope linked thereto. As such, the scaffold protein can be a synthetic poly(amino acid), which can be a homopolymer or heteropolymer, for example, a homopolymer such as poly(lysine), or can be a naturally occurring protein, or peptide portion thereof, for example, a viral coat protein such as a Hepatitis B core protein. Although the methods and compositions of the invention are exemplified using a scaffold protein, it will be recognized that any other molecule that provides a means to maintain the three dimensional structure of a selected epitope can be used herein. For example, a molecule for maintaining the three dimensional structure of a selected epitope could be a polynucleotide, such as DNA, RNA and the like, or a molecule could be a peptidomimetic, or any molecule that provides a covalent bond or bonds that maintains three dimensional structure, such as a linker molecule or a molecule within the selected epitope, such as an amino acid or side group.

A scaffold protein is exemplified herein by the Hepatitis B core (HBc) protein (SEQ ID NO:34, encoded by SEQ ID NO:33; see, also, GenBank Acc. Nos. 5542358 and 16930309, respectively, each of which is incorporated herein by reference), which provides the additional advantage that a plurality of HBC proteins can be induced to self assemble into a viral-like particle (VLP). Thus, an advantage of using a scaffold protein such as a viral coat protein, wherein a plurality of immunogenic peptide comprising the scaffold protein can be assembled to form a complex, is that a plurality of such scaffold proteins, each of which contains a selected epitope (i.e., the plurality of candidate immunogenic peptides), can be assembled to form a virus-like particle, thus presenting a clustered plurality of the epitopes. A further advantage of assembling a plurality of immunogenic peptides is that the epitopes of the plurality of immunogenic peptides can all be the same, can include some epitopes that are the same and some that are different, or can all be different. Where the immunogenic peptides of a plurality include epitopes that are different, the epitopes can be different epitopes of the same target protein, or can be epitopes of different target proteins.

A selected epitope can be linked directly to the scaffold protein, or can be linked via a linker moiety. A linker moiety can be any molecule capable of linking a selected epitope directly to a scaffold protein and can include, for example, an amino acid residue or peptide residue, such as one or more oligo(glycine) linkers or oligo(glycine-serine-alanine) linkers (see, e.g., Table 4, below). An advantage of linking the selected epitope to the scaffold protein via a linker moiety is that the size of the linker moiety can be varied, thus providing a means to obtain a variety of polypeptides, each of which include a selected epitope linked to a scaffold protein, wherein the selected epitope assumes different conformations and, therefore, providing an opportunity to identify a polypeptide in which the selected epitope is in a conformation corresponding to the conformation of the epitope in the native target protein (i.e., a candidate immunogenic peptide). In one embodiment, the linker moiety is a peptide such that the polypeptide containing the linker moiety, selected epitope, and scaffold protein is a fusion protein, which can be prepared using chemical peptide synthesis methods or can be expressed from a polynucleotide encoding the polypeptide.

A method of the invention also includes detecting that antibodies induced by a candidate immunogenic peptide can selectively bind to the target protein, thereby identifying an immunogenic peptide representative of a structural element of the target protein. Antibodies can be induced by a peptide and characterized for specificity by any known method. For example, a peptide (i.e., candidate immunogenic peptide) can be contacted with a cell or administered to a subject such that it can effect the subjects immunogenic activity. As such, the peptide generally will be administered in an amount, typically as a priming dose followed some time later by one or more booster doses, intradermally, subcutaneously, or intramuscularly, and if desired, formulated in a composition that includes an immunoadjuvant such as Freund's complete or incomplete adjuvant. Antibodies induced by a candidate immunogenic peptide can further be isolated and characterized for cross-reactivity with and specificity to a target protein. Antibodies of known specificity for a candidate immunogenic peptide can be tested for cross-reactivity with a selected epitope naturally present in a native target protein. For example, a target protein can be tested in ELISA assays for such cross-reactivity. A candidate immunogenic peptide capable of inducing antibodies that selectively bind the target protein is thereby identified as representative immunogenic peptide.

A target protein, for which an immunogenic peptide is identified according to a method of the invention, can be a protein of an infectious organism. For example, the infectious microorganism can be a bacterium that causes anthrax (e.g., Bacillus anthracis). Anthrax occurs in three main forms: cutaneous, gastrointestinal, and pulmonary. If diagnosed early enough, infection can be treated with antibiotics; but symptoms are not always apparent in time for antibiotic treatment to be effective. Thus, a means of conferring immune protection, such as by vaccination, can be important for protecting individuals at risk of exposure. Where the target protein is a protein of the bacterium Bacillus anthracis, the target protein can be an anthrax protective antigen (PA; SEQ ID NO:30; see, also, GenBank Acc. No. 2914135, which is incorporated herein by reference), which is encoded by SEQ ID NO:29 (GenBank Acc. No. 143280, which is incorporated herein by reference) or an anthrax lethal factor (LF; SEQ ID NO:32; see, also, GenBank Acc. No. 15974829, which is incorporated herein by reference), which is encoded by SEQ ID NO:31 (GenBank Acc. No. 15031488, which is incorporated herein by reference; Note: encodes four additional N-terminal amino acids as compared to SEQ ID NO:32), or both PA and LF.

Anthrax protective antigen (PA) is the dominant antigen in both natural and vaccine-induced immunity to anthrax infection. PA is a major component of the Anthrax Vaccine Absorbed (AVA) (ref 10), the vaccine currently licensed in USA. It is also essential for host cell intoxication in combination with either lethal factor (LF) or edema factor (EF), producing lethal toxin or edema toxin, respectively. Anthrax PA contains the host cell receptor binding site and facilitates the entry of the toxin complex into the host cell. The crystal structure of native PA has been elucidated and shows that PA includes four distinct and functionally independent domains. Domain 1 is divided into domains 1 a, including amino acids 1 to 167, and lb, including amino acids 168 to 258; domain 2 including amino acids 259 to 487; domain 3 including amino acids 488 to 595; and domain 4 including amino acids 596 to 735. Cell intoxication is thought to occur when full length PA binds to the cell surface receptor via domain 4, which contains the host cell receptor binding site. On binding to the host cell receptor, the N-terminal amino acids (1 to 167, i.e., domain 1a) of domain 1, which contains a furin protease cleavage site, are cleaved off, exposing the LF or EF binding site located in domain 1b and the adjacent domain 3. Domains 2 and 3 then form part of a heptameric pore on the cell surface, the LF or EF binds to its receptor, and the whole toxin complex undergoes receptor-mediated endocytosis into the cell. After acidification of the endosome, the toxin is translocated into the cell cytosol, where it exerts its cytotoxic effect. Thus, inhibition of the binding and entry of the toxin complex, particularly lethal toxin, into the host cell can be important for preventing infection.

The humoral response to PA component of anthrax toxin is a major determinant of both naturally occurring and vaccine-induced immunity (ref 11). The major role of a B cell response to PA in immunity against anthrax infection was confirmed by a study demonstrating a correlation between protection against inhalation anthrax and anti-PA Ig titers (refs 12, 13). Recently, a single dose of PA combined with various adjuvants was shown to provide significant protection (>90%) against inhalation anthrax infection in the rhesus macaques (ref 14). A study of passive protection of animals with antibodies (ref 15) showed that anti-PA serum (67% survival) protected better than serum from animals vaccinated with AVA (33%, ref 10) or live attenuated Sterne vaccine (10%, ref 16). These observations suggest that protective antigen can raise a toxin-neutralizing response, and can also confer protection against bacterial infection, making it an ideal vaccine candidate (ref 12). While the mechanism of PA-induced protection against anthrax infection is not clear, essential cross-reactivity of anti-PA polyclonal antisera with anthrax spores, and increased spore uptake by macrophages with anti-PA mAbs has been demonstrated (ref 10).

In addition to anti-PA antibodies, immunity to other exposed antigens plays an important role in protection against different strains of anthrax (refs 17, 18, 19, 20). A recent study of DNA-based anthrax vaccine candidates demonstrated that LF antigen is even more immunogenic than PA and produced an immune response that lasted much longer than the response to the PA antigen (ref 21). A significant synergy between PA and LF B cell epitopes also was demonstrated. The combination of PA and LF immunogens induced up to five times more antibodies in mice compared to either gene alone, and confers improved protection (refs 21, 22). Moreover, a passive immunization study has demonstrated that, while anti-PA antibodies play a major role in protection against anthrax spore infection, anti-LF antibodies most effectively protected against the lethal toxin itself (ref 23).

Thus, the present also relates to an isolated peptide of a B. anthracis protective antigen. Such isolated peptides of the invention are exemplified by a peptide consisting of amino acid residues 606 to 705 of SEQ ID NO:30; amino acid residues 606 to 735 of SEQ ID NO:30; amino acid residues 606 to 706 of SEQ ID NO:30; amino acid residues 606 to 704 of SEQ ID NO:30; amino acid residues 606 to 734 of SEQ ID NO:30; amino acid residues 607 to 703 of SEQ ID NO:30; amino acid residues 606 to 732 of SEQ ID NO:30; amino acid residues 604 to 707 of SEQ ID NO:30; amino acid residues 606 to 730 of SEQ ID NO:30; or amino acid residues 606 to 733 of SEQ ID NO:30.

The term “peptide” or “polypeptide” is used broadly herein to mean two or more amino acids linked by a peptide bond. If desired, a peptide of the invention, such as an immunogenic peptide can be modified, for example, to increase the ability of the peptide to act as an immunogen or a toleragen, to increase the stability of the peptide in a subject or other medium, or for any other purpose as desired. For example, the peptide can be modified by glycosylation, which can be effected by linking a carbohydrate moiety to a reactive side chain of an amino acid of the peptide or by including one or a few additional amino acids at the N-terminus or C-terminus of the peptide and linking the carbohydrate moiety to the additional amino acid. The linkage can be any linkage commonly found in a glycoprotein, for example, an N-linked or O-linked carbohydrate to an asparagine residue or a serine residue, respectively, or can be any other linkage that conveniently can be effected.

The invention also provides an isolated antibody that selectively binds such a peptide, provided the antibody does not substantially bind a peptide comprising amino acid residues 596 to 735 of SEQ ID NO:30; amino acid residues 679 to 693 of SEQ ID NO:3; amino acid residues 703 to 722 of SEQ ID NO:30, or amino acid residues 671 to 721 of SEQ ID NO:30. In this respect, it should be recognized that the requirement that an antibody of the invention specifically bind a peptide of the invention, but not bind the specified PA domains, or a polypeptide comprising such a domain (e.g., a full length PA polypeptide or a PA protein, only applies to an isolated antibody raised against a peptide of the invention; antibodies having such cross-reactivity are induced upon immunization of a subject with a composition of the invention and contribute to protection of the subject against PA function and anthrax. As such, antiserum, for example, isolated from a subject immunized with a composition of the invention, is encompassed within the present invention, as is a fraction of such antiserum containing the induced antibodies.

As used herein, the term “antibody” is used in its broadest sense to include polyclonal and monoclonal antibodies, as well as antigen binding fragments of such antibodies. The term “binds specifically” or “specific binding activity,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less. As such, Fab, F(ab′)₂, Fd and Fv fragments of an antibody that retain specific binding activity for an epitope of a GDF receptor, are included within the definition of an antibody.

The term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281 (1989), which is incorporated herein by reference). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995); each of which is incorporated herein by reference).

Methods for raising polyclonal antibodies, for example, in a rabbit, goat, mouse or other mammal, are well known in the art (see, for example, Green et al., “Production of Polyclonal Antisera,” in Immunochemical Protocols (Manson, ed., Humana Press 1992), pages 1-5; Coligan et al., “Production of Polyclonal Antisera in Rabbits, Rats, Mice and Hamsters,” in Curr. Protocols Immunol. (1992), section 2.4.1; each or which is incorporated herein by reference). In addition, monoclonal antibodies can be obtained using methods that are well known and routine in the art (Harlow and Lane, supra, 1988). Methods of preparing monoclonal antibodies well known (see, for example, Kohler and Milstein, Nature 256:495, 1975, which is incorporated herein by reference; see, also, Coligan et al., supra, 1992, see sections 2.5.1-2.6.7; Harlow and Lane, supra, 1988).

Monoclonal antibodies can be isolated and purified from hybridoma cultures by a variety of well established techniques, including, for example, affinity chromatography with Protein-A SEPHAROSE gel, size exclusion chromatography, and ion exchange chromatography (Coligan et al., supra, 1992, see sections 2.7.1-2.7.12 and sections 2.9.1-2.9.3; see, also, Barnes et al., “Purification of Immunoglobulin G (IgG),” in Meth. Molec. Biol. 10:79-104 (Humana Press 1992), which is incorporated herein by reference).

Antibodies of the invention also can be derived from human antibody fragments isolated from a combinatorial immunoglobulin library (see, for example, Barbas et al., METHODS: A Companion to Methods in Immunology 2:119, 1991; Winter et al., Ann. Rev. Immunol. 12:433, 1994; each of which is incorporated herein by reference). Cloning and expression vectors that are useful for producing a human immunoglobulin phage library can be obtained, for example, from STRATAGENE Cloning Systems (La Jolla, Calif.).

An antibody of the invention also can be derived from a human monoclonal antibody. Such antibodies are obtained from transgenic mice that have been “engineered” to produce specific human antibodies in response to antigenic challenge. Methods for obtaining human antibodies from transgenic mice are described, for example, by Green et al., Nature Genet. 7:13, 1994; Lonberg et al., Nature 368:856, 1994; and Taylor et al., Int. Immunol. 6:579, 1994; each of which is incorporated herein by reference.

The invention also provides an isolated polynucleotide encoding a peptide as recited above. In addition, the invention provides an isolated peptide consisting of amino acid residues 17 to 153 of SEQ ID NO:30; amino acid residues 261 to 454 of SEQ ID NO:30; or amino acid residues 487 to 594 of SEQ ID NO:30, as well as polynucleotides encoding such peptides, and an isolated antibody that specifically binds such an isolated peptide, provided the antibody does not bind substantially to a polypeptide comprising SEQ ID NO:30. Antibodies of the invention are useful, for example, for identifying and isolating a peptide of the invention from a sample such as a sample in which the peptide has been synthesized or expressed.

The term “polynucleotide” is used broadly herein to mean a sequence of two or more deoxyribonucleotides or ribonucleotides that are linked together by a phosphodiester bond. As such, the term “polynucleotide” includes RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the term “polynucleotide” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide of the invention can contain nucleoside or nucleotide analogs, or a backbone bond other than a phosphodiester bond (see above).

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

Where a polynucleotide encodes a peptide, for example, a polypeptide comprising a selected epitope linked to a scaffold protein, the coding sequence generally is contained in a vector and is operatively linked to appropriate regulatory elements, including, if desired, a tissue specific promoter or enhancer. The encoded peptide can be further operatively linked, for example, to peptide tag such as a His-6 tag or the like, which can facilitate identification of expression of the agent in the target cell. A polyhistidine tag peptide such as His-6 can be detected using a divalent cation such as nickel ion, cobalt ion, or the like. Additional peptide tags include, for example, a FLAG epitope, which can be detected using an anti-FLAG antibody (see, for example, Hopp et al., BioTechnology 6:1204 (1988); U.S. Pat. No. 5,011,912, each of which is incorporated herein by reference); a c-myc epitope, which can be detected using an antibody specific for the epitope; biotin, which can be detected using streptavidin or avidin; and glutathione S-transferase, which can be detected using glutathione. Such tags can provide the additional advantage that they can facilitate isolation of the operatively linked peptide or peptide agent, for example, where it is desired to obtain a substantially purified peptide corresponding to a proteolytic fragment of a myostatin polypeptide.

As used herein, the term “operatively linked” or “operatively associated” means that two or more molecules are positioned with respect to each other such that they act as a single unit and effect a function attributable to one or both molecules or a combination thereof. For example, a polynucleotide sequence encoding a peptide of the invention can be operatively linked to a regulatory element, in which case the regulatory element confers its regulatory effect on the polynucleotide similarly to the way in which the regulatory element would effect a polynucleotide sequence with which it normally is associated with in a cell. A first polynucleotide coding sequence also can be operatively linked to a second (or more) coding sequence such that a fusion protein can be expressed from the operatively linked coding sequences. The fusion protein is a polypeptide, in which the two (or more) encoded peptides are translated into a single polypeptide, i.e., are covalently bound through a peptide bond; or can be translated as two discrete peptides that, upon translation, can operatively associate with each other to form a stable complex. A fusion protein generally demonstrates some or all of the characteristics of each of its peptide components.

The present invention also relates to a composition, which includes at least a first epitope of a target protein, for example, a peptide of the invention, operatively linked to at least a first heterologous molecule. A composition of the invention generally is formulated in a physiologically acceptable solution and, if desired, can further contain one or more immunoadjuvants, for example, one or more cytokines, Freund's complete adjuvant, Freund's incomplete adjuvant, alum, or the like. Generally, where the composition contains one or more cytokines, the cytokines have an activity that is the same as or complements the inflammatory activity of the peptide of the invention. The composition also can contain any immunoadjuvant, including an immunostimulant or, if desired, an immunosuppressant, which can modulate the systemic immune response of an individual. Suitable substances having this activity are well known in the art and include IL-6, which can stimulate suppressor or cytotoxic T cells, and cyclosporin A and anti-CD4 antibodies, which can suppress the immune response. Such compounds can be administered separately or as a mixture with a vaccine of the invention.

Thus, in one embodiment, the composition includes at least a first peptide of the invention operatively linked to at least a first heterologous molecule. For example, the first peptide can be a peptide consisting of amino acid residues 606 to 705 of SEQ ID NO:30; amino acid residues 606 to 735 of SEQ ID NO:30; amino acid residues 606 to 706 of SEQ ID NO:30; amino acid residues 606 to 704 of SEQ ID NO:30; amino acid residues 606 to 734 of SEQ ID NO:30; amino acid residues 607 to 703 of SEQ ID NO:30; amino acid residues 606 to 732 of SEQ ID NO:30; amino acid residues 604 to 707 of SEQ ID NO:30; amino acid residues 606 to 730 of SEQ ID NO:30; or amino acid residues 606 to 733 of SEQ ID NO:30; wherein one or more of such peptides, which can be the same or different, is operatively linked to at least a first heterologous molecule.

The heterologous molecule, which is operatively linked to the peptide, can be any molecule, including, for example, a heterologous peptide, a peptidomimetic, a polynucleotide, a small organic molecule, or a combination of such molecules. In one aspect, at least a first heterologous molecule is a linker moiety, for example, a peptide linker such as an oligo(amino acid) (e.g., an oligo(glycine) linker or a (glycine-serine-alanine)N linker (see, e.g., Table 4). In another aspect, at least a first heterologous peptide, which is operatively linked to an epitope, particularly a peptide of the invention, is a scaffold protein, which provides conformational stability to the peptide. The scaffold protein can be a naturally occurring protein, or a peptide portion thereof, for example, a viral coat protein such as a Hepatitis B core protein, or a peptide portion thereof such as peptide portion set forth as amino acid residues 1 to 77 of SEQ ID NO:34 and a peptide portion set forth as amino acid residues 80 to 149 of SEQ ID NO:34, wherein the epitope/peptide is operatively linked at one terminus (e.g., the N-terminus) to amino acid residue 77 and at the other terminus (e.g., the C-terminus) to amino acid residue 80 of SEQ ID NO:34.

In still another aspect, the composition comprises an epitope or a peptide of the invention operatively linked to at least a first linker molecule, for example, a first and second linker molecule positioned at each terminus of the epitope/peptide, and further operatively linked, via the linker molecule(s), to a scaffold protein. For example, where the scaffold protein comprises two peptide portions of a viral coat protein such as amino acid residues 1 to 77 of SEQ ID NO:34 and amino acid residues 80 to 149 of SEQ ID NO:34, the epitope/peptide can be operatively linked to amino acid residue 77 via a first linker moiety and operatively linked to amino acid residue via a second linker moiety, wherein the first linker moiety and the second linker moiety are the same or different. In yet another aspect, the components of a composition of the invention are peptides, and the composition comprises a fusion protein, which, conveniently, can be encoded by and expressed from a polynucleotide.

A composition of the invention can further include at least a second heterologous molecule (e.g., a linker and a scaffold protein); a second and at least a third heterologous molecule (e.g., a first linker, a second, linker, and a scaffold protein); a second, a third, and at least a fourth heterologous molecule (e.g., a first linker, a second linker, a first portion of a scaffold protein, and a second portion of a scaffold protein), which can be in operative linkage.

As such, in one aspect, a composition of the invention comprises an immunogenic peptide representative of a structural element of a target protein. For example, the composition can include, in operative linkage, a first portion of a scaffold protein, a first peptide linker, a peptide of the invention, a second peptide linker, and a second portion of the scaffold protein (e.g., from N-terminus to C-terminus). Such a composition is exemplified herein by the immunogenic composition having an amino acid sequence as set forth in SEQ ID NO:36, which includes, from N-terminus to C-terminus, a first portion of a scaffold protein (amino acid residues 1 to 77 of SEQ ID NO:34), a first linker moiety (triglycine), a peptide of the invention (amino acid residues 606 to 704 of SEQ ID NO:30), a second linker moiety (triglycine), and a second portion of a scaffold protein (amino acid residues 80 to 149 of SEQ ID NO:34).

In another aspect, the first (or other) heterologous molecule of a composition of the invention comprises a tag operatively linked to a peptide of the invention. The tag can be any type of tag useful for detecting the presence of the peptide of the invention, for isolating the peptide, or the like. For example, the tag can be a peptide tag such as oligo(histidine) tag (e.g., His-6; 6×His), or can be a small organic molecule such as biotin, or the like. The tag further can be a detectable label. There are many different labels and methods of labeling known to those of ordinary skill in the art. Examples of the types of labels which can be used in the present invention include a fluorescent moiety, a luminescent moiety, or a chemiluminescent moiety. Those of ordinary skill in the art will know of other suitable labels for binding to the heterologous molecule, or will be able to ascertain such, using routine experimentation.

In another embodiment, a composition of the invention comprises a plurality of polypeptides, each polypeptide of the plurality comprising an immunogenic peptide representative of a structural element of a target protein, particularly a polypeptide comprising a peptide of the invention operatively linked to a viral coat protein. In one aspect of such a composition, the polypeptides of the plurality are assembled to form a virus-like particle.

A composition of the invention can be present in a solid form, for example, in a lyophilized form or a crystallized form, or can be in a solution, which can be an aqueous or non-aqueous solution, depending on the physico-chemical characteristics of the composition. In addition, the composition can be formulated for administration to a subject, for example, to stimulate antibodies against a target protein such as an anthrax protein. As such, the composition can further include other reagents useful for stimulating the immune response, for example, an adjuvant, or otherwise beneficial to the subject receiving the composition, for example, a therapeutic agent or a nutrient.

Where a composition of the invention is a fusion protein, the present invention further relates to a polynucleotide encoding the composition. A polynucleotide of the invention is exemplified by SEQ ID NO:35, which encodes a composition (SEQ ID NO:36) comprising a first portion of a scaffold protein (amino acid residues 1 to 77 of SEQ ID NO:34), a first linker moiety (Gly-3), a peptide of the invention (amino acid residues 606 to 704 of SEQ ID NO:30), a second linker moiety (Gly-4), and a second portion of a scaffold protein (amino acid residues 80 to 149 of SEQ ID NO:34).

The polynucleotide can be contained in a vector, which can be a cloning vector, thus providing a means to prepare a desired amount of the polynucleotide encoding the composition or to manipulate the polynucleotide; or can be an expression vector, thus providing a means to produce the encoded fusion protein. For example, the vector can be a viral vector, particularly a viral vector derived from a virus having a desired host or cell type specificity (e.g., a retrovirus, an adenovirus, a herpesvirus, or an adeno-associated virus).

The polynucleotide, which can, but need not be contained in a vector, also can be contained in a matrix that facilitates manipulation of the polynucleotide or introduction of the polynucleotide into a cell, for example, a matrix useful for forming liposomes or microbubbles. A polynucleotide of the invention, which can encode a polypeptide including a selected epitope linked to a scaffold protein, or can encode an immunogenic peptide, can be contained in a vector, which can facilitate manipulation of the polynucleotide, including introduction of the polynucleotide into a target cell. The vector can be a cloning vector, which is useful for maintaining the polynucleotide, or can be an expression vector, which contains, in addition to the polynucleotide, regulatory elements useful for expressing the polynucleotide and, where the polynucleotide encodes a peptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a promoter sequence, which can provide constitutive or, if desired, inducible expression of the encoding polynucleotide. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993; each of which is incorporated herein by reference).

Viral expression vectors can be particularly useful for introducing a polynucleotide into a cell. Viral vectors provide the advantage that they can infect host cells with relatively high efficiency and can infect specific cell types. For example, a polynucleotide encoding an epitope, or a polypeptide comprising a selected epitope linked to a scaffold protein, or an immunogenic protein can be cloned into a baculovirus vector, which then can be used to infect an insect host cell, thereby providing a means to produce large amounts of the encoded prodomain. The viral vector also can be derived from a virus that infects cells of an organism of interest, for example, vertebrate host cells such as mammalian, avian or piscine host cells. Viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference).

When retroviruses, for example, are used for gene transfer, replication competent retroviruses theoretically can develop due to recombination of retroviral vector and viral gene sequences in the packaging cell line utilized to produce the retroviral vector. Packaging cell lines in which the production of replication competent virus by recombination has been reduced or eliminated can be used to minimize the likelihood that a replication competent retrovirus will be produced. All retroviral vector supernatants used to infect cells are screened for replication competent virus by standard assays such as PCR and reverse transcriptase assays. Retroviral vectors allow for integration of a heterologous gene into a host cell genome, which allows for the gene to be passed to daughter cells following cell division.

A polynucleotide, which can be contained in a vector, can be introduced into a cell by any of a variety of methods known in the art (Sambrook et al., Molecular Cloning: A laboratory manual (Cold Spring Harbor Laboratory Press 1989); Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

A composition of the invention can be prepared for administration to a subject by mixing the immunogenic peptide or peptides with physiologically acceptable carriers. In one embodiment a composition of the invention is a vaccine. Such carriers will be nontoxic to recipients at the dosages and concentrations employed. Ordinarily, the preparation of such compositions entails combining the components of the composition, including a particular immunogenic peptide, with saline, buffers, antioxidants such as ascorbic acid, low molecular weight (less than about 10 residues) polypeptides, proteins, amino acids, carbohydrates including glucose or dextrans, or chelating agents such as EDTA, glutathione and other stabilizers and excipients. Such compositions can be in suspension, emulsion or lyophilized form and are formulated under conditions such that they are suitably prepared and approved for use in the desired application.

A physiologically acceptable carrier can be any material that, when combined with an immunogenic peptide or a polynucleotide of the invention, allows the ingredient to retain biological activity and does not undesirably disrupt a reaction with the subject's immune system. Examples include, but are not limited to, any of the standard physiologically acceptable carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed., Mack Publishing Co., Easton Pa. 18042, USA).

For administration to a subject, a peptide, or an encoding polynucleotide, generally is formulated as a composition. Accordingly, the present invention provides a composition, which generally contains, in addition to the peptide or polynucleotide of the invention, a carrier into which the peptide or polynucleotide can be conveniently formulated for administration. For example, the carrier can be an aqueous solution such as physiologically buffered saline or other solvent or vehicle such as a glycol, glycerol, an oil such as olive oil or an injectable organic esters. A carrier also can include a physiologically acceptable compound that acts, for example, to stabilize the peptide or encoding polynucleotide or to increase its absorption. Physiologically acceptable compounds include, for example, carbohydrates, such as glucose, sucrose or dextrans, antioxidants, such as ascorbic acid or glutathione, chelating agents, low molecular weight proteins or other stabilizers or excipients. Similarly, a cell that has been treated in culture for purposes of the practicing the methods of the invention, for example, synovial fluid mononuclear cells, dendritic cells, or the like, also can be formulated in a composition when the cells are to be administered to a subject.

It will be recognized to the skilled clinician that the choice of a carrier, including a physiologically acceptable compound, depends, for example, on the manner in which the peptide or encoding polynucleotide is to be administered, as well as on the route of administration of the composition. Where the composition is administered under immunizing conditions, i.e., as a vaccine, it generally is administered intramuscularly, intradermally, or subcutaneously, but also can be administered parenterally such as intravenously, and can be administered by injection, intubation, or other such method known in the art. Where the desired modulation of the immune system is tolerization, the composition preferably is administered orally, or can be administered as above.

A composition of the invention also can contain an additional reagent such as a diagnostic reagent, nutritional substance, toxin, or therapeutic agent, for example, a cancer chemotherapeutic agent. Preferably, the second reagent is an immunomodulatory agent, for example, an immunostimulatory agent such as a cytokine or a B7 molecule. In addition, where it is desired to stimulate an immune response, the composition can contain an adjuvant, for example, alum, DETOX™ adjuvant (Ribi Immunochem Research, Inc.; Hamilton Mont.), or Freund's complete or incomplete adjuvant. The addition of an adjuvant can enhance the immunogenicity of a peptide of the invention, thus decreasing the amount of antigen required to stimulate an immune response. Adjuvants can augment the immune response by prolonging antigen persistence, enhancing co-stimulatory signals, inducing granuloma formation, stimulating lymphocyte proliferation nonspecifically, or improving apposition of a T cell and an APC.

A composition comprising a peptide or polynucleotide of the invention also can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77, 1981, each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating material. Cationic liposomes, for example, also can be modified with specific receptors or ligands (Morishita et al., J. Clin. Invest., 91:2580-2585, 1993, which is incorporated herein by reference). In addition, a polynucleotide agent can be introduced into a cell using, for example, adenovirus-polylysine DNA complexes (see, for example, Michael et al., J. Biol. Chem. 268:6866-6869, 1993, which is incorporated herein by reference).

The present invention also relates to method of stimulating an immune response in a subject. Such a method can be performed, for example, by administering a composition of the invention, particularly an immunogenic peptide representative of a structural element of a target protein, or a polynucleotide encoding the composition, to a subject under conditions suitable for stimulating an immune response. Such conditions will depend, for example, on the material being administered. Where an immunogenic peptide is administered, it can be administered, for example, subdermally or mucosally, and can, but need not be formulated with an adjuvant, and can be administered as an initial dose and in booster doses, either or any of which can include an adjuvant. Where a polynucleotide encoding an immunogenic peptide is administered, the polynucleotide is administered such that it can enter cells in the subject and the encode immunogenic peptide can be expressed. In one aspect, the encoded immunogenic can further include a peptide sequence that directs translocation of the immunogenic to the cell surface and/or secretion of the immunogenic peptide from the cell such that it can stimulate an immune response in the subject.

A subject treated according to a method of the invention can be any subject in which it is desired to stimulate an immune response using an immunogenic peptide of the invention. As used herein, the term “stimulate an immune response” means that an immunogenic peptide of the invention is contacted with a cell or administered to a subject such that it can effect its immunogenic activity. As such, the composition, including an immunogenic peptide, generally will be administered in an immunogenic amount, typically as a priming dose followed some time later by one or more booster doses, intradermally, subcutaneously, or intramuscularly, and, if desired, formulated in a composition that includes an immunoadjuvant such as Freund's complete or incomplete adjuvant.

A method of the invention can be practiced with respect to a subject having, or predisposed or susceptible to, any condition in which it is desired to modulate an immune response, including a subject that has an immunologic disorder, or is susceptible or predisposed to an immunological disorder. The subject generally is a vertebrate subject, and particularly a mammal, including a domesticated animal such as a cat, a dog, or a horse; a farm animal such as an ovine, bovine or porcine animal; or a human. The immunological disorder can be a disorder of the immune system such as a condition in which the subject has not developed a sufficient immune response, for example, in response to an infectious disease or a cancer.

The total amount of a composition to be administered in practicing a method of the invention can be administered to a subject as a single dose, either as a bolus or by infusion over a relatively short period of time, and can be followed up with one or more booster doses over a period of time. The amount of the composition to stimulate an immune response in a subject depends on various factors including the age and general health of the subject, as well as the route of administration and the number of treatments to be administered. In view of these factors, the skilled clinician will know to adjust the particular dosage as necessary. In general, the formulation of the composition and the routes and frequency of administration are determined, initially, using Phase I and Phase II clinical trials.

Administration of a peptide or polynucleotide of the invention to a subject predisposed to, but not yet having developed, the disease can be accomplished by short term administration of one or more dosages of the composition sufficient to produce detectable increases, for example, antibodies specific for an immunogenic peptide. In one embodiment, the subject treated according to a method of the invention is a human subject, wherein the immune response in the subject can be stimulated to protect the subject from harm due to an infectious microorganism that expresses the target protein, or to ameliorate such harm following infection with the microorganism. In one aspect, the subject is a human subject infected with or susceptible to infection with B. anthracis, and administration of the immunogenic peptide generates a protective immune response in the subject against the signs and symptoms of anthrax. A method of the invention can further include isolating antibodies induced in a mammalian subject, from the subject, thus providing antiserum, or a fraction thereof containing antibodies, e.g., a fraction of isolated antibodies, which can be useful for a passive immunization procedure.

As discussed above, the methods of the invention are useful for identifying immunogenic peptides of a target protein having a known three dimensional structure, or of a target protein having a known amino acid sequence but an unknown three dimensional structure. In another embodiment, a method of the invention is used to identify an immunogenic peptide representative of a structural element of a target protein having a known amino acid sequence but an unknown three dimensional structure. Such a method can be performed, for example, by generating a molecular model of a three dimensional structure of the target protein based on the three dimensional structure of a homologous protein; selecting epitopes of the target protein based on the molecular model of the three dimensional structure of the target protein, thereby obtaining selected epitopes; detecting, in a molecular model of a chimeric polypeptide comprising at least one selected epitope having a constrained structure, an epitope having a three dimensional conformation corresponding to the molecular model of the three dimensional structure of the epitope in the target protein, thereby identifying a candidate immunogenic peptide representative of a structural element of the target protein; and detecting that antibodies induced by the candidate immunogenic peptide selectively bind to the target protein, thereby identifying an immunogenic peptide representative of a structural element of the target protein.

In addition to the applications discussed above, modeling systems are used for predicting three dimensional structure in a target protein where the amino acid sequence is known, but the three dimensional structure is unknown (see Examples 2 and 3). In such instances, the amino acid sequence of the target protein with an unknown three dimensional structures is used to identify homologous polypeptide sequences utilized in subsequent steps in modeling a target protein according to the invention.

Homology or identity is often measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more polypeptide sequences, refer to two or more sequences or subsequences that are the same or have a specified percentage of amino acid residues that are the same when compared and aligned for maximum correspondence over a comparison window or designated region as measured using any number of sequence comparison algorithms or by manual alignment and visual inspection. For the purposes of molecular modeling according to the present invention, a protein is considered homologous if at least about 20%, usually at least about 25%, and particularly at least about more than 30%, homology exists between the protein and a target protein.

For a given target protein sequence, a “template” homologous protein having a known three dimensional structure generally can be identified (e.g., in PDB). The best template protein or proteins are identified as having the highest similarity score, as defined by any one of the accepted homology search methods, e.g. BLAST2 (Altschul et al., J. Mol. Biol. 215:403-410, 1990), PSI-BLAST (ref 24), Smith and Waterman, Adv. Appl. Math. 2:482, 1981, by the homology alignment algorithm of Needleman and Wunsch, J. Mol. Biol. 48:443, 1970, each of which is incorporated herein by reference). Alternatively, a homology search can be performed using the ZEGA alignment algorithm as implemented in the ICM™ software suite molecular modeling package.

To build a homology model for the target antigenic proteins, algorithms integrated in the ICM™ software suite molecular modeling package, which has a long track record of successful homology modeling applications (refs 4, 5, 6, 7, 9, 25). For the first step of the procedure, optimal alignment between the target sequence and the template sequence is found, based on modified ZEGA algorithm (ref 26), which allows overlapping gaps in both sequences, a unique feature important for correct alignment of loop regions. Alignment scoring also accounts for structural features of the template, including surface accessibility of aligned residues, secondary structure and disulfide bond patterns.

Given an accurate alignment, a second step of the ICM™ software suite procedure (ref 9) performs initial placement of the target polypeptide chain onto the template. Initial placement is followed by more elaborated energy-based modeling, including global energy optimization of loops and side chains. This third step includes conformational modeling of protein side chains and loops in the ICM™ software suite relies on internal coordinate definition of the molecular object, as well as computationally efficient Biased Probability Monte Carlo (BPMC) optimization (ref 1). An extended force field includes surface terms, electrostatics with the boundary element solution of the Poisson equation (ref 2), and side chain entropy terms. Several independent runs of this optimization program will be performed to verify convergence of the BPMC procedure. Finally, quality of structural models are assessed by the ICM™ software suite calculation of normalized residue conformational stress (ref 27). If significant stress is found in some parts of the protein, conformational modeling of these regions will be repeated with reformulated structural constraints. The parameters used with the above algorithms may be adapted depending on the sequence length and degree of homology studied. In some embodiments, the parameters may be the default parameters used by the algorithms in the absence of instructions from the user.

The predicted structure of the target protein is then used for obtaining selected epitopes, as described above. In one embodiment, selected epitopes are epitopes of the target protein that have been selected based on the three dimensional structure of the molecular model of the target protein as being positioned on the target protein such that they are particularly available to contact with an antibody. Selected epitopes can include, for example, peptide portions of the target protein that are on a surface of the target protein, including peptide portions of the target protein that can be bound by an antibody in the native protein; and/or synthetic peptides based on discontinuous peptide portions of a target protein that are in special proximity in the protein, and or/peptide portions of the target protein that are predicted to be soluble in aqueous solution.

A method of the present invention also includes identifying a candidate immunogenic peptide representative of a structural element of the target protein. An immunogenic peptide is identified by comparing a chimeric polypeptide to a selected epitope of the molecular model of the target protein. Thus, in one embodiment, a “candidate immunogenic peptide” is a chimeric polypeptide including a selected epitope having a constrained structure, wherein the epitope has a three dimensional conformation corresponding to the three dimensional conformation of the epitope in a target protein. The three dimensional conformation of the epitope in a target protein refers to the conformation of the epitope in the molecular model of the target protein, and is predicted to correspond to the epitope as it is present in a native or naturally-occurring target protein.

The molecular modeling systems described above are used in comparing a chimeric polypeptide to a selected epitope of the molecular model of the target protein. Computer programs for carrying out the activities necessary to detect candidate immunogenic peptides are well known in the art. For example, methodology for epitope modeling can be implemented in the ICM™ software suite from Molsoft, LLC (see above).

Accordingly, the invention provides an immunogenic peptide identified according to a method of the invention. In one aspect, the chimeric polypeptide and, therefore, an immunogenic peptide identified therefrom, is a fusion protein. In one aspect, the chimeric polypeptide, which can be a fusion protein, includes a plurality of linked selected epitopes, wherein the selected epitopes of the plurality are the same, are different, or include some that are the same and others that are different. The chimeric polypeptide can further include at least one linker peptide, which is linked to at least one selected epitope of the chimeric polypeptide.

The constrained structure of the selected epitope can be due, for example, to at least one disulfide bond in the chimeric polypeptide, to cyclization of the chimeric polypeptide (e.g., via a peptide bond formed between the C-terminus and N-terminus of the chimeric polypeptide or the selected epitope portion thereof; or via a bond formed between two reactive side chains of amino acid residues in the chimeric polypeptide); or due to linkage of the selected peptide to a scaffold protein, for example, a viral coat protein such as Hepatitis B core protein, or a poly(amino acid) such as poly(lysine). In another aspect of a method of the invention, the selected epitope is linked to a scaffold protein via a linker moiety, for example, a peptide linker.

A method of the invention can further include assembling a plurality of immunogenic peptides, in which the selected epitope is operatively linked to a viral coat protein, into a virus-like particle. The selected epitopes of the immunogenic peptides in such as assembled virus-like particle can be the same or different or a combination of some that are the same and others that are different.

A method of the invention also includes detecting that antibodies induced by a candidate immunogenic peptide can selectively bind to the target protein, thereby identifying an immunogenic peptide representative of a structural element of the target protein. Similar to above, antibodies can be induced by a peptide and characterized for specificity by any known method. For example, a peptide (i.e., candidate immunogenic peptide) can be contacted with a cell or administered to a subject such that it can effect the subjects immunogenic activity. Antibodies induced by a candidate immunogenic peptide can further be isolated and characterized for cross-reactivity with and specificity to a target protein. Antibodies of known specificity for a candidate immunogenic peptide can be tested for cross-reactivity with a selected epitope naturally present in a native target protein. For example, a target protein can be tested in ELISA assays for such cross-reactivity. A candidate immunogenic peptide capable of inducing antibodies that selectively bind the target protein is thereby identified as representative immunogenic peptide.

A target protein, for which an immunogenic peptide is identified according to a method of the invention, can be a protein of an infectious microorganism, particularly a protein involved in infectiveness of microorganism or a deleterious effect caused by the microorganism. The infectious microorganism can be, for example, a protozoan such as Entamoeba histolytica, a yeast such as Candida albicans, or a bacterium such as Shigella flexneri, Yersinia pestis, or Serratia marcescens.

As such, an infectious microorganism can be the bacterium Shigella flexneri. Shigellosis (bacillary dysentery) is a disease that is endemic throughout the world, representing a major concern in terms of morbidity and mortality in developing countries where conditions of sanitation are poor, personal hygiene practices are often inadequate, and malnutrition exists. Shigella flexneri is the most common species worldwide. Approximately 450,000 estimated cases of Shigellosis occur in the US each year, and 150 million cases are seen in developing countries, where it is primarily a pediatric disease. More then 1 million deaths are recorded annually, and developing an effective vaccine against Shigellosis is one of the top 3 priorities of the World Health Organization.

Humans are the natural host and reservoir for Shigella. Shigella infections are readily transmitted from person-to-person by direct contact involving fecally contaminated fingers, and less commonly, by contaminated food and water. The severity of the illness in infected individuals varies, ranging from a mild watery diarrhea to severe dysentery (high fever, toxemia, scanty stools that contain blood). Shigella infections are a major problem in refugee and military populations. The current world political climate underscores the inherent need for an effective vaccine in crisis refugee management. It should be emphasized that as yet, no good Shigella vaccine is available. Vaccines to date have been predicated on attenuated microbial strains for oral vaccine delivery.

Infection is initiated by ingestion of the pathogen. Within any population of Shigella organisms there exist some bacteria that are acid-resistant and able to survive in an environment of pH 2 for up to 4 hr. Thus, without buffering of gastric contents, the dose causing infection in humans may be as low as 10 organisms. The cardinal virulence property of Shigella is traditionally considered to be their ability to invade epithelial cells, particularly the colonic epithelium in the small intestine. Virulence components including the pic gene are required for effective colonization and degradation of the mucin protecting the intestinal lining. The organism invades and transverses the M cell, and thereby infects the underlying epithelium. A pathogenicity island (PAI, the ipa/mxi-spa locus) located within a 200 kilobase virulence plasmid is necessary and sufficient for Shigella entry into epithelial cells via macropinocytosis. The key to insertion is the secretion of a flagella-like structure that inserts a pore into the cell membrane. The pore is a complex of IpaB and IpaC proteins. Epithelial cell infection leads to cell lysis via apoptosis induced by IpaB, and induction of IL-1β secretion. This prompts a vigorous inflammatory response, and recruitment of inflammatory cells to the site of infection results in massive tissue destruction and manifests the bloody diarrhea characteristic of disease. Recovery is a balance between the rate of infection and phagocytic destruction of infected cells, and the ability to repair the damaged epithelium. In vulnerable younger patients, in the absence of antibiotics or electrolyte supplements, mortality can be very high.

Pic is known to be a member of the autotransporter family of bacterial proteins, so named because of their distinctive mechanism of transport through the gram-negative outer membrane. The proteins are secreted through the inner membrane by virtue of the Sec apparatus. Once in the periplasm, a dedicated C-terminal domain folds into a putative β-barrel and the surviving N-terminus is translocated through the pore of the barrel, which remains lodged in the membrane. In most cases, the N-terminal moiety is released from the cell. The ability of the purified pic protein to cleave mucin was confirmed using mucin purified from the cecum of mice and also using purified bovine submaxillary mucin. The protein was also able to cleave both gelatin and casein in zymogram gels. A predicted catalytic serine at residue 258 of the protein was mutated, and the protein was found to lose protease and mucinase activity. Because of its mucinase activity, pic mucinase activity is promotes the penetration of intestinal mucus blanket and therefore colonization.

The pic locus has been detected present in nearly all S. flexneri 2a strains and also in the majority of enteroaggregative E. coli (EAEC) isolates. The set/pic locus has been found in 56% of the EAEC strains from diarrhea patients, but only 0.7% of EAEC from controls. Interestingly, the locus has also been found in some isolates of other E. coli pathotypes, including two ETEC strains and a DAEC (all of these patients had diarrhea). The picket-positive EAEC also has been associated with diarrheal disease in Mexican children. As such, a target protein can be, for example, an autotransporter of a bacterium such as an autotransporter as set forth in Table 1 (e.g., a Shigella autotransporter such as a Shigella flexneri protease involved in colonization (pic) gene product.

In another embodiment, an infectious microorganism can be, for example, a bacterial protein having an enzymatic activity such as a phospholipase activity, e.g., the activity of a Yersinia murine toxin as expressed by a Yersinia species such as Yersinia pestis. Yersinia pestis is the highly virulent organism that causes plague in a wide range of animals, including humans. Infection results in a high rate of mortality. Plague is endemic in regions of North and South America, Africa, China and Asia and there are 1000-2000 human cases annually. Individuals living or traveling in endemic areas and laboratory workers handling the bacterium are at risk of disease. In the United States, plague is enzootic in the western states and has been identified as a risk at 25 military installations in these states.

Plague is normally transmitted from infected rodents to humans via the bite of an infective flea. The bubonic form of the disease results when the bacteria migrate to regional lymph nodes where the bacteria multiply and cause swelling to generate the characteristic and painful bubo. If untreated the disease will progress to septicemia and may progress to secondary pneumonic plague. The pneumonic form of the disease typically results in a productive cough which releases a highly contagious aerosol and may lead to the spread of primary pneumonic plague, the most severe form of the disease. Pneumonic plague is difficult to diagnose and is almost always fatal unless antibiotic treatment is started at the first sign of disease.

The high virulence of Yersinia pestis is due to a complex array of factors encoded by both the chromosome and three plasmids. The plasmids are the 9.5 kb pPCP (pesticin, coagulase, plasminogen activator), the 70-75 kb pCD (calcium dependence) and the 100-110 kb pMT or pFra, which encodes a murine toxin and the capsular protein, F1. This plasmid is unique for Y. pestis.

Yersinia murine toxin (Ymt) is highly toxic to mice and rats, but less active in guinea pigs, rabbits, dogs and monkeys. The toxin is not secreted and is associated primarily with the cell membrane or the cytoplasm until cell death and lysis. Ymt has an LD50 for mice of 0.2 to 3.7 μg. It may act as a β-adrenergic antagonist that directly blocks β-adrenergic receptors after in vivo activation and cause circulatory collapse. The exact role of the toxin in pathogenesis has not been established. Ymt expression is higher at 26° C. than at 37° C., and Ymt is required for the survival of Y. pestis in the flea vector.

The gene for Ymt has been cloned and sequenced, and encodes a 61-kDa protein. Ymt shows strong homology to a phospholipase D (PLD) precursor encoded by Streptomyces antibioticus. Ymt also shows homology to eukaryotic enzymes of the PLD superfamily. Four regions have been identified as common to eukaryotic PLDs, and Ymt shows significant homology to two of these regions, suggesting that Ymt and PLD might utilize a common catalytic mechanism.

The present invention also relates to an isolated peptide, consisting of a peptide as set forth in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or SEQ ID NO:11 (Table 5), each of which is a peptide of a Shigella flexneri pic gene product. In addition, the present invention relates to an isolated peptide, consisting of a peptide as set forth SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, or SEQ ID NO:28 (Table 6), each of which is a peptide of a Yersinia pestis murine toxin (Ymt). Also provided is a polynucleotide encoding a peptide consisting of any of SEQ ID NOS:1 to 28, wherein the polynucleotide can, but need not, be contained in a vector (e.g., a viral vector) and/or in a matrix (e.g. liposomes or microbubbles).

The present invention further relates to a composition, which includes at least a first peptide of the invention operatively linked to at least a first heterologous molecule. In one embodiment, the composition includes a plurality of peptides of the invention, wherein the peptides of the plurality are linked to each other, for example, a plurality of two, three, four, five, six, etc., peptides, which can be the same or different or a combination thereof. In one aspect, the first heterologous molecule can be a linker moiety, for example, a peptide linker or biotin, or can be any heterologous molecule, including any heterologous peptide. In another aspect, the first heterologous peptide comprises a scaffold protein, for example, a poly(amino acid) such as poly(lysine), or a viral coat protein such as a Hepatitis B core (HBc) protein (e.g., a first portion of the HBc protein consisting of amino acid residues 1 to 77 of SEQ ID NO:34 and a second portion of the HBc protein consisting of amino acid residues 80 to 149 of SEQ ID NO:34, wherein the peptide of the invention is operatively linked to amino acid residue 77 and to amino acid 80 of SEQ ID NO:34. In another embodiment, the composition includes a plurality of peptides of the invention, wherein each peptide of the plurality is operatively linked to a viral coat protein. In one aspect of this embodiment, the plurality of peptides is assembled, via the viral coat protein component, to form a virus-like particle.

A composition of the invention can first include a second heterologous molecule, a third heterologous molecule, a fourth heterologous molecule, etc. In one aspect, the composition includes at least a second heterologous molecule, and the first heterologous molecule is a linker moiety or a scaffold protein or a portion thereof. For example, composition can include a first heterologous molecule that is a linker moiety and a second heterologous molecule that is a scaffold protein (or a portion thereof), and the linker moiety can operatively link the peptide of the invention (or other selected epitope) to the scaffold protein. In another example, the composition can include, in operative linkage, a first portion of a scaffold protein, a first peptide linker, the peptide of the invention (or other selected epitope), a second peptide linker, and a second portion of the scaffold protein.

The first (or other) heterologous molecule in a composition of the invention also can be a tag, such as a peptide or other small molecule tag, which can, but need not, be a detectable label or provide a means for identifying and/or isolating the composition. The first (or other) heterologous molecule also can be any other molecule of interest, including, for example, a carrier protein; a peptide that directs transport of the composition to an intracellular compartment or to a cell surface, or directs secretion of the composition out of a cell.

In one embodiment, a composition of the invention is an immunogenic composition, which comprises an immunogenic peptide representative of a structural element of a target protein. As such, the composition can be formulated for administration to a subject, for example, by including an adjuvant or other agent that may be desirable for stimulating an immune response. Accordingly, the invention further relates to a method of stimulating an immune response in a subject by administering such an immunogenic composition, or, where the immunogenic composition comprises a polypeptide, a polynucleotide encoding the composition, to a subject under conditions suitable for stimulating an immune response. The immune response stimulated in the subject can be a protective immune response, and the subject can be any subject in need of such an immune response, for example, a mammalian subject such as a human subject.

The following examples are intended to illustrate but not limit the invention.

### EXAMPLE 1

### Identification and Characterization of Anthrax Immunogenic Peptides

Protection with PA. Numerous studies have demonstrated that the humoral response to PA component of anthrax toxin is a major determinant of both naturally occurring and vaccine-induced immunity (ref 11). The major role of a B-cell response to PA in immunity against anthrax infection has been confirmed in studies demonstrating a correlation between protection against inhalation anthrax and anti-PA Ig titers (refs 12, 13). A single dose of PA combined with various adjuvants has been shown to provide significant protection (>90%) against inhalation anthrax infection in the rhesus macaques (ref 14). Passive protection of animals with antibodies (ref 15) has shown that anti-PA serum (67% survival) protects better than does serum from animals vaccinated with AVA (33%) or Sterne (10%). These observations suggest that protective antigen not only can raise a toxin-neutralizing response but can also confer protection against bacterial infection, making it an ideal vaccine candidate (ref 12). While the mechanism of such PA-induced protection against anthrax infection is not clear yet, a possible explanation comes from a recent study by Welkos et al., where they have demonstrated essential cross-reactivity of anti-PA polyclonal antisera with anthrax spores, as well as correlated increased spore uptake by macrophages with anti-PA mAbs (ref 10).

During the last years, more data appeared validating PA as candidate recombinant vaccine (refs 28, 29). One of the studies, from the Williamson group, (ref 29) demonstrated protection in mice induced by microsphere-associated PA, administered both by mucosal or parenteral routes. Another study by the same group (ref 30) has shown that the carboxy-terminal domain (Domain 4) contains the dominant protective epitopes of PA and confers protection against anthrax infection in mice. The latter result validates our choice of PA Domain 4 as a target conformational epitope. Feasibility of mucosal delivery route for PA-epitope covered microparticles suggests similar possibility for the delivery platform, employed in our project, which displays PA epitopes on a surface of self-assembling virus like particles (VLPs).

Protection with Lethal Factor (LF). In addition to anti-PA antibodies, immunity to other exposed antigens plays an important role in protection against different strains of anthrax (refs 17, 18, 19, 20). A recent study of DNA-based anthrax vaccine candidates has demonstrated that LF antigen appears to be even more immunogenic and produces an immune response that lasts much longer than does the response to the PA antigen (ref 21). This study has also shown significant synergy between PA and Lethal Factor B-cell epitopes. The combination of PA and LF immunogens induces up to five times more antibodies in mice compared to either gene alone, and confers improved protection (refs 21, 22). Moreover, a very recent passive immunization study by Kobiler et. al has demonstrated that, while anti-PA antibodies play a major role in protection against anthrax spore infection, it is anti-LF antibodies that most effectively protect against the lethal toxin itself (ref 23).

Therefore, it would be beneficial to use this recently discovered synergy between PA and LF in future vaccine formulations. However, combination of these two full-length proteins forms a functional PA-LF toxin, and its use in a recombinant vaccine would be highly hazardous (refs 31, 32). Instead of using whole protein toxins, small structural domains of PA and LF were used in combination with an effective VLP delivery system to maximize protective immunity without compromising safety.

A. Design of Epitopes of Anthrax Protective Antigen (PA)

The following describes design of linked domain of anthrax protective antigen into Hepatitis B core scaffold protein.

1. Mapping Functional and Physical Properties of PA, Selection of Surface Target

Annotation of PA protein structure, based on available antibody mapping of the Protective Antigen protein, pointed to Domain 4 as a most potent target for antibody response. Analysis of PA domain structure and physical annotation of the protein surface also indicated that Domain 4 is highly accessible to antibodies. Unlike other domains of PA, Domain 4 has a compact β-sandwich structure with a well-defined hydrophobic core, which assures its high structural stability. Moreover, Domain 4 also has the least surface contact with other PA domains (˜750 Å²). All these qualities make PA Domain 4 an attractive target as a candidate conformational epitope.

An extensive analysis of immunogenicity and protective efficacy of overlapping regions of the PA polypeptide (ref 30), has confirmed that Domain 4 contains most dominant protective epitopes of PA. Moreover, it also confirms our prediction that Domain 4 can be detached from PA and used as an effective stand-alone antigen. Based on these computational and experimental data, epitope design efforts focused on Domain 4 of the PA (Table 2).

The known antigenic region in Domain 4, covers two flexible loops of PA, a “large loop” (residues 704-722) and a “small loop” (residues 679-693). As our analysis shows, the “large loop” lies on the interface between Domain 4 and domain 2. When Domain 4 in detached, one shall expect significant conformational changes in the loop. Indeed, global optimization of the “large loop” in the detached Domain 4 suggests that original conformation of the loop is not preserved.

On the other hand, conformational modeling of the “small loop” (residues 679-693) clearly demonstrates that its shape is well preserved between detached and attached forms of the domain. The loop backbone is relatively rigid and constitutes a part of a larger conformational epitope, covering a substantial part of the Domain 4. Based on this information, residues 679 to 693 of the “small loop” were selected as the primary immunodeterminant, and the surface patch within 10 A from the center of the small loop as a surface target for 3D structural epitope design.

2. Identification of Conformationally Stable 3D Structural Epitopes

Stability analysis was performed for all four domains of PA to identify each domain's most stable “core” part, capable of preserving the epitope 3D conformation. A panel of 900 polypeptides was listed for each domain, with all possible N- and C-terminal deletions (up to 30 residues from each end). Relative fold stability of these polypeptides was evaluated with the ICM procedure based on free-energy calculations as described (refs 2, 27). This Energy Strain function, which includes conformational strain, implicit entropy, and implicit solvation terms, was specifically developed and normalized to study the stability of polypeptide chains (refs 27, 2). The top ten most stable sub-folds for Domain 4 are presented in Table 3, along with the top results for the other PA domains.

As evident from the first four columns in Table 3, the Energy Strain predicts optimal stability for both the full domain PA4{606:735} and for the truncated PA4{606:705} form with one α-strand deleted. The surface energy component (column 4) of the function is slightly higher for the truncated form, PA4{606:705}, but its stability is probably compensated for by deletion of a “large loop” which otherwise destabilizes the overall fold. An important advantage of the truncated PA4{606:704} fold is the small distance between its termini (column 5), optimal for VLP insertion, while this distance is too large in the full PA4. Based on the above analysis, the PA4{606:704} domain was selected as a prime candidate 3D epitope for VLP insertion.

3. Rational Design of PA4-HBc Chimeric Protein

Selection of Optimal Insertion Region

An analysis of the HBc MIR region was performed to identify optimal insertion sites for structural epitopes. The protein residues between G73 and G94 are disposable, providing a variety of possibilities for design (ref 33). All of the combinations of insertion points in this range were taken and a simple energy check was performed by fast minimization of (Gly)₄ (SEQ ID NO:37) peptides attached to these residues. This analysis predicted some energetic preference to minimal deletions in the MIR region and, therefore, only a 3 residue loop of HBc{77:79} was used for replacement. This insertion site differs by one amino acid from the traditional insertion site HBc{76:80}, but was expected to yield a slightly more stable design.

Rational Design of Epitope-VLP Chimera

A panel of candidate linkers to insert PA have been compiled using both oligo-Gly linkers and a Gly-Ser-Ala repeat (ref 34). Linker lengths of between 3 and 10 amino acids were chosen, with the difference between the lengths of the two linkers not exceeding 2 amino acids (see Table 4). The full-length 3D models of chimeric protein were built from PA and HBc models according to the following “formula”: PA_HBc(XY)=HBc{1:76}+Linker1+PA{606:704}+Linker2+HBc{80:142}. For each of the candidates, a global energy optimization of linker conformations was performed, considering flexibility of linkers themselves and two residues flanking the linkers from both sides. A special “local” version of ICM Monte Carlo procedure has been employed, wherein energy optimization is performed in specified flexible linkers (or loops) and the rigid domains of the protein are allowed to move as rigid bodies (ref 35). Energy function was calculated in the same way as in loop modeling (see C. 1), and global energy optimization was performed until convergence. The best 100 alternative conformations for each polypeptide were collected and saved in the conformational stack.

Evaluate Fold Stability and Self-Assembly of Chimeric VLPs

VLP self-assembly and fold stability have been assessed by building partial VLP for the chimeric protein and checking the multimer for steric clashes between protein subunits. To build 36-mers, representing all contacts between chimeric VLP subunits, the Icosahedral Symmetry transformation matrix from PQS Protein Quaternary Structure Database at the EBI was used. To enforce ideal symmetry of the VLP particle, Torsion variables in the individual subunits in the multimer were “synchronized”. This natural approximation greatly reduces the number of free variables in the system and allows reproducible and conclusive global energy optimization. The energy of the 36-mer was calculated for all candidate conformations including both intramolecular (conformational energy of linkers) and intermolecular (van der Waals, electrostatic, hydrogen bonding and solvation terms). The results of these global optimization runs are presented in Table 4 for the top 10 candidates. Presented are predictions of chimeric PA-VLP stability. Variations were calculated from four different independent runs.

As illustrated in Table 4, the conformational strain is low in the linkers of the energy-minimized PA-VLP monomers, and does not significantly vary between different linker lengths. This can be explained by sufficient flexibility of poly(G) linkers and possibility of large-scale rearrangements of PA4 domain in a monomer. On the other hand, when these large-scale movements are restricted by formation of a multimer or VLP, the energy difference is more pronounced, and provides the basis for candidate selection. The major preference is for Linker1 to be one amino acid shorter than Linker2, and the optimal length of linkers is about 3-4 amino acids. The poly(Gly) linkers were generally preferred over GSA repeats. Five top candidate polypeptides with the lower conformational energy in multimers were selected for experimental testing. The best VLP chimera will be serving as leads for further improvements.

4. Expression and Purification of Chimeric VLPs

Synthetic genes encoding C-terminal truncated forms of Hepatitis B core antigen (HBc) and hybrid PA-HBc polypeptides were produced by contract gene synthesis (Blue Heron Biotechnology). 430-450 bp genes for HBc and 750-800 bp genes for candidate hybrid PA-HBc proteins were Taq polymerase-amplified from plasmid DNA and cloned directly into a pTrcHis2-TOPO expression vector by TOPO TA cloning (Invitrogen). This vector encodes a C-terminal peptide containing the c-myc epitope and a 6×His tag. These tags add ˜4 kDa to the size of the expressed protein. Recombinant vectors were transformed into TOP 10 E. Coli and transformants screened by restriction digest and PCR analysis. Sequences of selected clones were confirmed by contract DNA sequencing (Qiagen Genomics).

Pilot-scale expression cultures of selected transformants were grown at 37° C. to an OD-600 of 0.5-1.0, then induced with IPTG for 4 hr and cell lysates analyzed for protein expression by SDS-PAGE and western blot under reducing conditions. Monoclonal antibodies specific for the two regions of HBc antigen located on either side of the PA insert detected a single band at a molecular weight appropriate for the recombinant protein plus the added tags. Monoclonal antibody specific for the C-terminal His tag also recognized a single band at the same molecular weight, demonstrating full-length read-through protein expression.

E coli expression of hybrid PA-HBc was scaled up to 300 ml in flasks and the cell pellets processed for recovery and purification of His-tagged recombinant protein. Cells were lysed with BugBuster™ extraction reagent (Novagen) with added benzonase nuclease, lysozyme and protease inhibitors, followed by centrifugation. Both clarified cell lysate and insoluble material were analyzed by SDS-PAGE and western blot. Most of the expressed PA-HBc protein was found in the insoluble cell pellet, which was further processed to recover a purified inclusion body fraction. This strategy was successful in concentrating the recombinant protein and removing the majority of the contaminating E. coli material. Inclusion body fractions were dissolved in 8M Urea, 0.3M NaCl, 0.2% nonionic detergent in phosphate buffer pH 7.5 and freeze-thawed once before further purification by immobilized metal affinity chromatography (IMAC).

Various affinity resins were used to purify His-tagged recombinant under denaturing conditions, including Ni-NTA, Ni-IDA and Talon cobalt resins. The Talon Co²⁺ resin (Clontech) provided the best results for eliminating non-specific E. coli proteins, so this resin was chosen for PA-HBc purification. All binding and wash steps included 8M Urea and 5 mM 2-ME. Recombinant protein was eluted from the affinity column with 200 mM imidazole in 8M Urea, 0.2% nonionic detergent, 0.3M NaCl in phosphate buffer pH 7.5. Samples were collected from all purification fractions and analyzed by SDS-PAGE. A single protein band at ˜34 kDa was reproducibly eluted from the immobilized metal affinity chromatography (IMAC) affinity columns, with a purity estimated at ˜95%. Fractions containing eluted protein were pooled and concentrated by centrifugal filtration, then buffer exchanged into PBS and re-natured on PD-10 columns. The yield was estimated to be 1-2 mg purified protein per liter of E. coli culture.

5. Rabbit Immunization with Chimeric VLPs Induced PA-Specific Antibodies

Samples of purified, re-natured PA-HBc protein are analyzed for self-assembly into core particles by size exclusion chromatography and electron microscopy. PA-VLPs were tested in ELISA assays for recognition by anti-PA antibodies specific for the cell receptor binding domains of PA antigen.

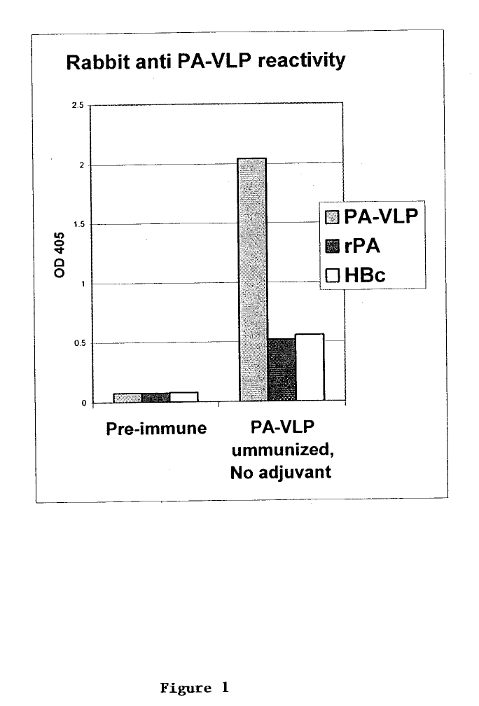

New Zealand White rabbits were immunized intramuscularly (IM) with 200 μg of chimeric PA-VLP protein, without adjuvant, and boosted intramuscularly (i.m.) with 100 μg, without adjuvant, at weeks 1, 4 and 10. Antisera from week five was tested by ELISA and Western blot for reactivity to chimeric PA-VLP, recombinant Hepatitis B core protein and recombinant anthrax PA. Results shown in FIG. 1 show that these rabbits responded not only to the PA-VLP antigen, but also to Hepatitis B core and to anthrax PA. Both ELISA data and Western blot data showed cross-reactivity of PA-VLP induced antibodies against recombinant Protective Antigen (rPA).

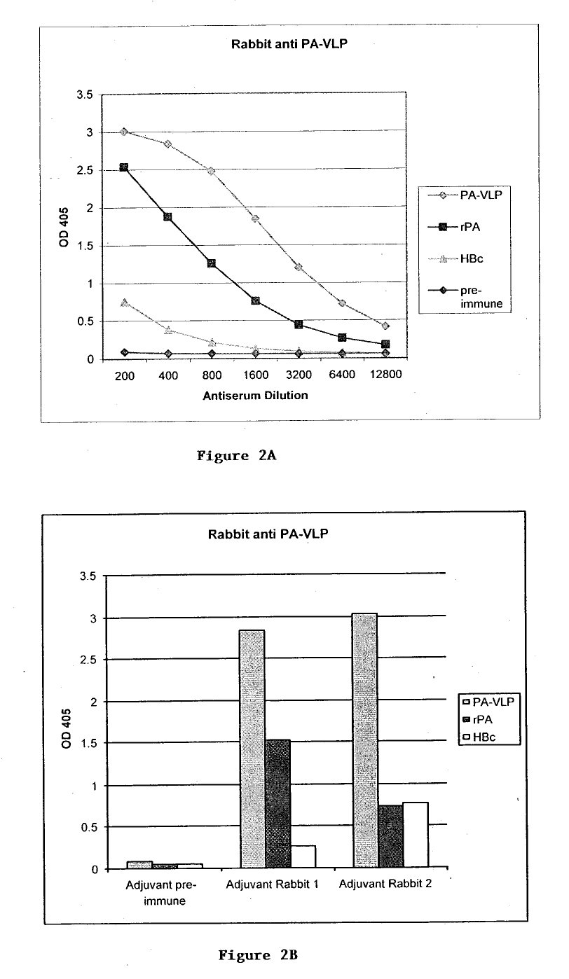

Additional rabbits were immunized IM with 200 μg of chimeric PA-VLP protein in complete Freund's adjuvant and boosted IM with 100 μg protein in Freund's incomplete adjuvant at weeks 1, 4 and 10. Antisera from week 11 cross-reacted strongly with anthrax rPA in ELISA assays (FIG. 2).

B. Identification and Design of Lethal Factor (LF) Epitopes on VLPs

Antibodies, induced in response to LF antigen, confer protection against lethal toxin action (ref 23) and work in synergy with anti-PA humoral response to protect against anthrax infection (ref 21). The computational platform developed in Phase I will be used to identify and design most potent LF epitopes (i.e., epitopes of SEQ ID NO:32), encoding polynucleotides of which can be genetically inserted in HBc virus-like particles.

1. Identify Optimal B-Cell Epitopes in LF

Completing all-atom 3D model of LF protein. A complete all-atom molecular model of the Lethal Factor protein is required both for sequence-structure annotation and for energy-based conformational modeling. Two X-ray structures (ref 36) of LF are available in the Protein Database, in apo-form (PDB: 1J7N) and bound to the N-terminal sequence of Mapkk2 (PDB: and 1JKY). Both 3D structures have a number of omissions, compared to full-length LF protein, which has 781 residues. These features, not represented by the PDB structure, may be highly relevant to the LF immunogenicity. The LF molecular object will be completed by building an ICM molecular tree, automatically assigning atom types, reconstructing hydrogens and missing loops, and then using extensive conformational modeling to predict the most probable configurations of these loops.

Annotate the LF structural model. Having the complete molecular object built, LF physical properties and surface features, which are important for LF-antibody recognition and which correlate with immunogenicity, will be annotated. First, highly exposed features protruding from the protein surface will be identified as putative “primary” epitopes (ref 37) that can play a role in immunogenicity. This analysis is performed by calculating so called “protrusion index” (ref 3 8) and also by using the original method based on calculations of solvent exposed surface-to-volume ratio for all protein fragments.

Also, the analytical molecular surface (skin) of the protein (refs 3, 39) will be found and accurate electrostatic solvation energy mapping of this surface by the boundary element method implemented in the ICM package (ref 2) will be performed. Mapping hydrophobic regions as well as donor and acceptor groups exposed to the surface of the protein will be accomplished. Finally, protein backbone flexibility of the model using crystallographic B factor will be annotated.

The structural model of LF protein will be annotated with the functional information, including active sites and protein binding sites, which often correlate with immunogenicity. LF comprises four domains. Domain I binds to PA in the process of membrane translocation. Domain IV contains a zinc metalloprotease active site, while domains II, III and IV together create a long deep groove that holds the 16-residue N-terminal tail of MAPKK-2 before cleavage.

Available experimental data on LF-specific monoclonal antibodies will help identify neutralizing epitopes in LF. An extensive study of LF immunogenic characterization is presented in a classic work by Little et al. (ref 40). In this work, sixty-one monoclonal antibodies to the LF protein have been characterized for specificity, antibody subtype, and ability to neutralize lethal toxin. Only three monoclonal antibodies (10G3, 2E7, and 3F6) neutralized lethal toxin in rats. In a macrophage cytolysis assay, monoclonal antibodies 10G3, 2E7, 10G4, 10D4, 13D10, and 1D8, but not 3F6, were found to neutralize lethal toxin. Five out of six monoclonal antibodies that neutralized lethal toxin in the macrophage assay did so by inhibiting the binding of LF to the cell attached PA fragment. In contrast, monoclonal antibody 1D8, which was also able to neutralize lethal toxin activity after lethal factor was prebound to cell-bound protective antigen, only partially inhibited binding of LF to protective antigen. Monoclonal antibody 3F6 did not inhibit the binding of LF to protective antigen. A competitive-binding enzyme-linked immunosorbent assay showed that at least four different antigenic regions on LF were recognized by these seven neutralizing hybridomas. The anomalous behavior of 3F6 suggests that it may induce a conformational change in LF.

Some of these functional and immunogenic features can be explained by domain structure of the LF protein. One striking feature is an almost complete structural independence of N-terminal Domain I, which has only 850 A contact area with the rest of the protein. Domain I, responsible for PA binding and translocation of the toxin, is attached to the catalytic part by a single α-helix, suggesting that large-scale spatial rearrangements of this domain in course of translocation. It is likely that some of the antibodies (i.e. mAb 1D8 and/or 1F6 mentioned above; ref 40) can bind to LF between Domain I and the catalytic domains and prevent these conformational changes, thus hindering LF translocation to host cell. The completed and annotated 3D model of LF will be used to further localize the most probable “primary” determinants within these immunogenic regions and to estimate the position of the whole antibody recognition site.

Identification of surface targets. Knowledge of LF immunogenic and functional features described above provides a basis for the rational choice and analysis of candidate anti-LF structural epitopes. The protein surface of LF will be systematically sampled with ˜1000 overlapping patches defined as the surface within a certain radius from the “center” of the patch. The effective radius of these candidate surface targets corresponds to the average dimension of antibody-epitope recognition sites. All candidate targets on the LF surface will be ranked according to the following criteria and correlated with immunogenicity: 1) overlap with the experimentally described antigenic regions (ref 4 1); 2) epitope sequence conservation measured by number of variable residues (ref 42); 3) overlap with protruding surface features on PA surface (ref 38); 4) complexity of geometrical shape and electrostatic features of the surface. Since protection against LF largely correlates with inhibition of PA-LF translocation complex and animal protection, the PA binding site in LF Domain I represents the major interest as a neutralizing epitope. Monoclonal antibodies, targeting this epitope, can compete for PA binding and thus prevent LF transport into host cells. As an alternative, possible antigenic sites on the in the LF protein can be predicted, specific antibody binding to which can interfere with large-scale rearrangements of Domain I, required for translocation.

2. Rationally Design LF-HBc Chimeric Proteins

A stable sub-fold of the LF protein, which would accurately represent a candidate surface target, will be identified. The major part of a target surface and its native conformation must be preserved when the structural epitope is detached from the rest of the polypeptide chain. The size of an optimal sub-fold is governed by requirements for VLP self-assembly and should not exceed 250 residue range (refs 33, 43). The procedures described below were successfully used to design other candidate vaccines, including PA epitopes and PA-VLP hybrids.

Estimation of fold stability and selection of 3D structural epitopes. Relative fold stability of selected polypeptides will be assessed with an ICM procedure, based on fast and accurate evaluation of free energy of the polypeptide model. This free energy function, which includes conformational strain, implicit entropy and implicit solvation terms, was specifically developed and normalized to study stability of polypeptide chains (ref 27). A similar free-energy function has also been recently used in successful ab initio folding of a 23-residue peptide (ref 2). The free-energy evaluation procedure in ICM is fast enough (˜1000 per CPU hour) that stability for all possible continuous sub-sequences of LF protein having lengths between 50 and 250 residues can be checked in a reasonable time.

Based on the above analysis, polypeptide sequences will be chosen that satisfy requirements for (i) structural fold stability, (ii) maximum coverage of the surface target area, and (iii) optimal spatial distance between the first and the last residue in the polypeptide fold. The latter selection criteria will be important on the next step of epitope-VLP chimera design. Based on these criteria, the best candidate 3D structural epitope for each of the three major immunogenic regions of the anthrax Protective Antigen will be selected, and these three representative targets will be used in epitope-VLP design.

Rational design of LF epitope-HBc chimera. Design is performed similar to design of optimal PA-HBc protein. The structure-based design of epitope-HBc chimeric proteins focuses on optimization of folding of the protein, assembly of icosahedral VLP, and display of the surface target antigen. First, a panel of candidate linkers having either a traditional oligo-Gly sequence content, or a Gly-Ser-Ala repeat, another widely used linker (ref 34), is compiled. All linker lengths between 3 and 10 amino acids will be considered, and the difference between the lengths of the two linkers will be limited to 2 amino acids, thus having about 50 polypeptides to test for each sequence content.

Second, conformational modeling will be performed to evaluate fold stability and self-assembly of chimeric polypeptides. For each candidate LF-HBc design, 36-mers are built that represent all contacts between chimeric VLP subunits, using Icosahedral Symmetry transformation matrix from PQS Protein Quaternary Structure Database at the EBI. To enforce ideal symmetry of the VLP particle, the torsion variables will be “synchronized” in the individual subunits in the multimer. This natural approximation greatly reduces the number of free variables in the system and allows reproducible and conclusive global energy optimization. The energy of the 36-mer will be calculated for all candidate conformations, including both intramolecular (conformational energy of linkers) and intermolecular (van der Waals, electrostatic, hydrogen bonding and solvation terms). The global optimization allows large-scale rearrangements of the foreign epitope domain and HBc domain and conformational changes in two flexible linkers connecting domains. This type of calculation is highly facilitated in ICM internal coordinate representation where domains of the protein are considered as rigid bodies, and only a small portion of the polypeptide, corresponding to the flexible linkers, is optimized (ref 35). Finally, the same global optimization procedure will be used to evaluate self-assembly of “mosaic” VLPs that include both PA-HBc and LF-HBc protein subunits. Several symmetry patterns of mosaic VLP, representing both assembly of PA-HBc/LF-HBc heterodimers and homodimers of each chimeric protein, will be taken into consideration.

About five candidate polypeptides with the lowest VLP energy will be selected for experimental testing. It is possible that the best LF-HBc candidates for homogeneous and “mosaic” particles will be predicted to be different. In this case, candidates from both groups will be selected for further evaluation.

C. Test and Optimize PA-VLP and LF-VLP Designs

Building upon the pilot testing of the first PA-VLP design in Phase I, a more elaborate comparative study will be performed assessing folding, VLP assembly and specific immunogenicity for the initial and other PA-VLP and LF-VLP candidates predicted in silico.

1. Express and Purify the Five Top-Ranked PA-VLPs Candidates Designed in Phase I and the Five-Top Ranked LF-VLP Candidates; Compare Stability of the Particles

To express the additional 4 optimal PA-VLP constructs, small modifications in initial PA-HBc(G3*G4) design will be introduced by insertions or deletions in the DNA template, using splice overlap extension PCR. Genes for LF-VLP candidates will be synthesized as variants of the original HBc constructs, inserting coding sequence for the LF epitopes into the template at the MIR site. The new synthetic DNA constructs will be inserted in the E. coli expression vector, and purified as described above. Purified recombinant PA and LF-HBc hybrids will be induced to polymerize and assemble into core particles by buffer exchange and the appropriate assembly into core particles will be monitored by Sepharose CL-4B gel filtration chromatography. The macromolecular nature of the material complexed into VLPs will be indicated by its near void volume elution. Samples of purified particles will also be examined by electron microscopy by contracting with an appropriate facility.

2. Assess Specific Antigenicity of Candidates in PA-VLP and LF-VLP Groups to Available Anti-PA and Anti-LF Sera, Respectively

Purified wild-type and recombinant HBc particles will be analyzed for protein yield, purity, and the presence of Hepatitis B core antigen (HBcAg) and target polypeptide epitopes using standard western blot and ELISA assays. Monoclonal antibodies against both PA and LF have been produced and characterized for specificity and ability to neutralize toxin activity (refs 40, 44). The epitopes for these antibodies have been identified by their ability to inhibit binding of PA to the cell receptor or to inhibit binding of LF to cell-bound PA. These antibodies will be used through Cooperative Research and Development Agreements to characterize the purified recombinant proteins by western blot and to confirm the structure of the epitopes presented on our recombinant PA-VLP and LF-VLP vaccines by ELISA assay.

Samples taken at various steps during protein expression, isolation and purification will be analyzed by SDS-PAGE and western blot to evaluate relative protein. Control binding by anti-HIS tag monoclonal antibody, and HBcAg monoclonal antibodies will verify a protein product of correct size is produced. Purified HBcAg will be electrophoreses as a positive control, along with purified recombinant PA and LF. Recombinant HBc containing PA and LF domains will be tested using corresponding monoclonal antibody panels.

Commercially available antibodies to the polyhistidine tag and to recombinant HBc antigen will be used in a sandwich ELISA format to capture recombinant HBc, and thus native recognition of PA and LF domains within the recombinant HBc molecules will be tested by reactivity to respective PA and LF antibodies.

The solubility of the recombinant HBc proteins, and thus their recovery from E. Coli, may be influenced by the inserted target epitope. If it is necessary to verify insertion of PA or LF epitopes in the recombinant HBc protein, peptide mapping can by used to identify the insertion of the epitope peptide in the appropriate location of the HBc protein.

3. Demonstrate Immunogenicity of PA-VLP and LF-VLP Formulations in Rabbits; Assess Cross-Reactivity of Rabbit Sera to Recombinant PA and LF

The ability of the recombinant HBc VLP to stimulate target-specific immunologic responses will be evaluated. Standard procedures for VLP assembly will be followed, and assembly determined by size-exclusion chromatography and electron microscopy. The percentage of recombinant protein excluded from P 100 chromatographic columns, measured by ELISA and spectrophotometrically, will be taken as appropriately folded VLP. Qualitative confirmation will be measured by scanning electron microscopy performed at a contract laboratory. Variation between assembly runs, and comparison between sequential purification runs, will determine the robustness of the purification and assembly process as a first stage in process development.

Rabbits will be immunized with either recombinant HBc particles or wild-type control particles (n=6). Antisera from test bleeds will be compared to pre-bleeds in ELISA assays, where either recombinant HBc particles or wild type control particles are attached to microtiter wells and reacted with rabbit antisera. Rabbit antibodies are then detected by enzyme-labeled anti-Ig and colorimetric substrate. Antibody titers from rabbits immunized with recombinant HBc particles will be compared with those from rabbits immunized with wild-type HBc particles to determine the effect that the inserted target epitope has on HBc immunogenicity.

4. Assemble Homogeneous and Mosaic PA-LF-VLP, Using Different Mixtures of Candidates from Both Groups; Verify their Specific Antigenicity and Immunogenicity

Using purified recombinant chimeric HBc protein, VLPs will be assembled and subjected to stability and immunogenicity testing. Standard procedures for VLP assembly will be followed (ref, Phase I report), and assembly will be determined by size-exclusion chromatography and electron microscopy. The percentage of recombinant protein excluded from P100 chromatographic columns, measured by ELISA and spectrophotometrically, will be taken as appropriately folded VLP.

Assembled VLPs will be tested for successful epitope presentation by reactivity with PA or LF specific monoclonal antibodies using an interference assay. The assay standard will be ELISA-based capture of PA/LF antibody against recombinant (non-assembled) HBc immobilized in microtiter plates. A titration of assembled VLP versus standard will allow quantitation of VLP-presented epitope, and analytical size exclusion chromatography of the ELISA supernatants, detecting rabbit antibody, will confirm the stability of assembled VLPs.

To assess the composition of mosaic VLPs comprised of dual recombinant PA and LF proteins, a titration of one recombinant protein relative to a fixed level of the second will be performed prior to VLP assembly. Composition will be assayed by ELISA, in which samples will be subjected to capture by PA or LF monoclonal antibody, and detection of the complementary epitope measured by binding of appropriate conjugated monoclonal antibody. This assay will be performed using both PA and LF monoclonal antibodies as capture reagents, and epitope binding relative to mass of VLP determined. Proportions of recombinant HBc will be chosen which, when mixed and assembled, bind equivalent molar proportions of monoclonal antibody.

The mosaic VLPs will then be used in immunogenicity studies in rabbits, as described above. Potency of VLP preparations will be measured as the reciprocal of antibody dilution showing signal above pre-bleed values. Variations between animals will be analyzed, and all data compared to the immunogenicity studies performed in above.

The value of mosaic VLPs, compared with homogeneous VLPs, which are mixed prior to final filling, is not established. Stability studies comparing mosaic VLPs relative to homogeneous VLP mixtures will be performed to determine benefit. Differential stability between PA and LF VLP, a mixture of separately assembled PA and LF VLP, and mosaic VLP will be the rationale for selection and evaluation in a product development scheme.

D. Formulate Vaccine and Perform Animal Immunogenicity and Protection Testing

1. Optimize and Scale-Up Production and Purification of the Most Potent PA-VLP, LF-VLP and PA-LF-VLP Chimeric Proteins

Current VLP expression levels correspond to about 3 mg/liter of E. coli culture, and expression has not been optimized for fermentation conditions. Our plan is to perform initial small scale (1 to 5 liter) fermentation culture determinations for recombinant HBc yield, and then to make a decision regarding contract manufacturing for scale-up productions. A number of contract facilities exist for E. coli based recombinant protein production (see, for example, Abgent, Inc.; Imgenex, Inc.), and several of these perform contract VLP assembly.

VLP produced by scale-up production and purification will be placed in short-term stability studies, comparing PBS formulation and cold storage to lypophilization and ambient storage. Accelerated stability determinations will be made by using a range of storage temperatures and plotting decay parameters. The ideal formulation of ambient, lyophilized product is our target. Initial stability assay parameters will be assembly and retention of in vitro immunological reactivity, and chosen samples will be taken to immunogenicity determinations.

2. Formulate PA-VLP and “Mosaic” PA-LF-VLP Candidate Vaccines

A major advantage of VLP for humoral immune stimulation is their potency in the absence of T-cell adjuvants. The intention is to follow industry precedence and to perform immunogenicity and stability studies using limited formulations of clinically approved intravenous salt solutions. The initial studies described above will be performed using standard research grade PBS, and barring negative findings, clinical grade formulations and alternatives will be researched for secondary studies.

Vehicle selection for immunogenicity studies will be based primarily on stability studies described above, given the extensive data collected to date validating PBS delivery of VLP. A single formulation will be used for initial immunogenicity studies described below.

3. Perform Immunization Dose Range Studies in Animals, Delivered via Alternate Routes

Mouse Immunogenicity Studies. Standard immunogenicity dose ranging studies will be performed in Balb/c inbred mice. Based on published findings, VLP are potent in both prime and boost stages of humoral responsiveness, and identically formulated for each stage.

Study cohorts will be organized in groups of n=10. Two routes of delivery will be initially studied, the intramuscular and the intranasal routes. For each route, animals will be organized to receive 3 doses of VLP as prime: 100 ng; 2 μg, and 40 μg, respectively. Each prime dose will be followed by 3 boosting regimens of 100 ng; 2 μg, and 40 μg, respectively. Control animals (n=10 per arm) will be injected with PBS vehicle only. This constitutes a total of 80 animals, for each arm of the study. Four arms are planned for the first phase: PA-VLP, LF-VLP, mosaic PA-LF-VLP, and a 50/50 mixture of PA-VLP and LF-VLP. All samples will be assembled and formulated just prior to injection.

Boosts will be performed at monthly intervals. Retro-orbital bleeds will be taken prior to boost. Sera will be measured for potency against PA or LF using ELISA based screening against both recombinant VLP antigen, and purified PA or LF purchased from commercial sources. Also, isotype distribution of PA/LF-specific antibodies is assessed, especially IgG vs. IgA titers. Control reactivities against HBcAg will be measured using monoclonal anti-HBcAg antibodies. Reactivity will be reported as reciprocal titer demonstrating signal above pre-bleed, and data reported for both cohort and individual responses. Following a final boost at 4 months, half of each group (n=5) will remain on study for an additional 6 months, during which persistence of humoral responsiveness will be monitored as described above.

Sera will be measured for potency against PA or LF using ELISA based screening against both recombinant VLP antigen, and purified PA or LF purchased from commercial sources. Control reactivities against HBc will be measured using monoclonal anti-HBcAg antibodies. Reactivity will be reported as reciprocal titer demonstrating signal above pre-bleed, and data reported for both cohort and individual responses.

4. Test VLP Protection Against Anthrax Lethal Toxin (LF+PA) Challenge

Preliminary animal protection studies will be performed using purified toxins and avoiding the complications of viable pathogen challenge. This can be accomplished by using bolus injection of Lethal Toxin (PA plus LF).

In Vitro Protection Assay. In vitro neutralization assays will be conducted to determine the ability of antisera to inhibit PA- and LF-induced mortality of J774A. 1 mouse macrophage cells (ATCC). Sera from PA-VLP, LF-VLP, mosaic PA-LF-VLP, 50/50 PA-VLP/LF-VLP and control-immunized animals will be serially diluted and incubated with 50 ng/ml PA and 40 ng/ml LF for 1 hour to allow neutralization to occur. Pre-incubated PA and LF will then be added to susceptible cells. Percent survival of cells will be determined by a calorimetric assay. The end point will be defined as the highest serum dilution that exhibits the approximate absorbance value of the control serum. The neutralizing antibody titer will be expressed as the reciprocal end-point dilution. Data will be analyzed by standard statistical methods. Most potent formulations will be taken into animal studies.

Challenge Trial. It is intended to use mice immunized in the dose ranging study described above as candidates in the lethal challenge model. Two weeks following final boost in each study, half of each cohort group will receive a lethal injection dose of PA/LF mixture, delivered i.v. The equivalent of approximately five (5) 50% lethal doses (LD₅₀), i.e., 60 μg of PA and 30 μg of LF, is used per mouse. Acute mortality is generally seen within 3 days. Protection will be assessed by determining the number of surviving animals/number of challenged animals. These studies can safely be performed using BSL-2 containment.

It is established that protective immunization can be obtained using either PA or LF as an immunizing reagent, hence there is much value in comparing serum titers against PA and LF in the multiply vs. singly antigen challenged cohorts. All animals will be sacrificed at 1 week post-challenge and gross necropsy performed to gather confirmatory pathology. These experiments will be outsourced to a contract pathology service.

Rabbit Protocols. Polyclonal antisera will be produced in 24 female, 1.4-2.7 kg, New Zealand White rabbits. This work will be performed as contract services through QED (San Diego Calif.), according to standard operating procedures (SOP #R102-1 “Rabbit Immunizations, Subcutaneous” and SOP#R101-1 “Rabbit Bleeding”). QED is a USDA-licensed facility with appropriate IACUC and OLAW oversight. Samples of purified chimeric PA-VLPs and wild-type control VLPs will be diluted in sterile PBS without adjuvant and injected i.m. or s.c. four times during a standard 60 day protocol. One pre-immunization and four post-immunization bleeds will be collected from each rabbit and antibody titers to the different VLP preparations will be measured by standard ELISA assays.

Veterinary care is provided by and health records are available from the facility performing this work. Animals are observed daily by animal care staff and veterinary staff is on call 24 hrs a day, year round. Animals are allowed quarantine and stabilization periods and are separated by species, source and project to control disease and zoonosis. Animals that show signs of illness are treated, or euthanized, at the veterinarian's discretion. Unnecessary pain/distress is not acceptable. All personnel working with the rabbits are on an approved protocol and are trained in the procedures they perform.

Rabbits to be used for this work are bred at the facility performing the immunizations, eliminating stress or discomfort associated with transport. Only animals that appear clinically healthy will be used for antisera production. Procedures will be performed on rabbits that are properly secured in a restraining device that allows normal breathing and minimizes discomfort, distress, pain and injury. Analgesic, anesthetic and tranquilizing drugs are not normally used for these procedures.

It may not be necessary to euthanize the rabbits at the end of this study, but if it becomes necessary, euthanasia will be performed in accordance with the recommendations of the 1993 AVMA Panel on Euthanasia. A drug such as Euthasol™ euthanizing agent containing 390 mg pentobarbital sodium and 50 mg phenytoin sodium will be administered at 1.0 ml per rabbit up to 10 pounds.

Mouse Protocols. Standard immunogenicity dose ranging studies will be performed in Balb/c inbred mice. Study cohorts will be organized in groups of n=1 0. Two routes of delivery will be initially studied, intramuscular and intranasal. For each route, animals will be organized to receive 3 doses of VLP as prime: 100 ng; 2 μg, and 40 μg respectively. Each prime dose will be matrixed with 3 boosting regimens of 100 ng; 2 μg, and 40 μg respectively. Control animals (n=10 per arm) will be injected with PBS vehicle only. This constitutes a total of 80 animals, for each arm of the study. Four arms are planned for the first phase: PA VLP, LF VLP, PA/LF mosaic VLP, and a 50/50 mixture of PA/LF VLP. All samples will be assembled and formulated in PBS just prior to injection.

Boosts will be performed at monthly intervals. Retro-orbital bleeds will be taken prior to boost. Sera will be measured for potency against PA or LF using ELISA based screening against both recombinant VLP antigen,- and purified PA or LF purchased from commercial sources. Control reactivities against HBc will be measured using monoclonal anti-HBcAg antibodies. Reactivity will be reported as reciprocal titer demonstrating signal above pre-bleed, and data reported for both cohort and individual responses.

Following a final boost at 4 months, half of each group (n=5) will remain on study for an additional 6 months, during which persistence of humoral responsiveness will be monitored by serum titer on monthly bleeds. At the end of the 6 month period, animals will receive a final boost followed in two weeks by sera collection and euthanasia to conclude the study. The remaining half of the animals will be used in a lethal toxin challenge model. (below).

Lethal Toxin Challenge (mouse). Preliminary animal protection studies will be performed using purified toxins and avoiding the complications of viable pathogen challenge. This can be accomplished by using bolus injection of Lethal Toxin (PA plus LF).

It is intended to use animals immunized in the dose ranging study described above as candidates in the lethal challenge model. 2 weeks following final boost in each study, half of each cohort group will receive a lethal injection dose of PA/LF complex (60 μg of PA and ˜30 μg of LF in PBS per mouse), delivered i.v. Acute mortality is generally seen within 3 days. These studies can safely be performed using BSL-2 containment, and our subcontractor is equipped and experienced in BSL-2 pre-clinical studies.

It is established that protective immunization can be obtained using either PA or LF as an immunizing reagent, hence there is much value in comparing serum titers against PA and LF in the multiply vs. singly antigen challenged cohorts. All animals will be sacrificed at 1 week post-challenge and gross necropsy performed to gather confirmatory pathology. These experiments will be outsourced to a contract pathology service (PAI).

### EXAMPLE 2

### Identification and Characterization of Shigella Immunogenic Peptides

This example illustrates the use of structural biology design principles to define key interventional epitopes for vaccine development against a class of pathogenic molecules produced by enteric bacteria. This method is exemplified by targeting the pic gene (protease involved in colonization) of Shigella flexneri, important in pathogenesis of dysentery. A panel of structural epitopes, within a related family of pathogenic molecules can provide a safe and broad vaccine component for many diarrheal and mucosal diseases.

A newly discovered family of enteric disease associated proteins share a common secretory pathway. These proteins are classified as “autotransporters”, based on their ability to self-direct membrane translocation and cleavage to produce a secreted mature protein. This is accomplished through structural motifs inherent in the precursor molecule that spontaneously form a membrane pore through which the mature domain of the molecule is translocated. Members of this family include the Shigella pic gene, the Neisseria secretory IgA protease, and the Helicobacter vacA toxin. with many other examples of toxins or biochemical properties linked to colonization or cell invasion.

The identification of common structural motifs utilized by this family of proteins allows development of a broad based vaccine strategy designed to block secretion of the disease-associated activities. Principles of computational homology modeling, in combination with phylogenetic comparisons, have predicted surface exposed structural epitopes for vaccine responses. Identification of these structural epitopes now allows immunogenicity testing and proof-of-principle studies to demonstrate that vaccine strategies can effectively block secretion and pathogenesis within this class of proteins.

Homology Modeling of the Shigella pic Gene

Bioinformatics analysis has linked the pic gene to the autotransporter family of secreted proteins. Structural homology modeling was performed using the gene sequence of the Shigella pic gene as input against the solved structures in the PDB (Protein Data Base). Significant homology was found to several members of the autotransporter family. Two of these family members have solved crystal structures, a secreted toxin from Bordetella pertactin, and an E. coli membrane porin. The homologies identified were primarily between the secreted domains of the pic gene with homology to the Bordetella pertactin toxin, or the beta-barrel domains of the E. coli membrane porin. Analyses with the known structures of the above mentioned proteins indicate show the probable structure of the pic gene based on the homology modeling.

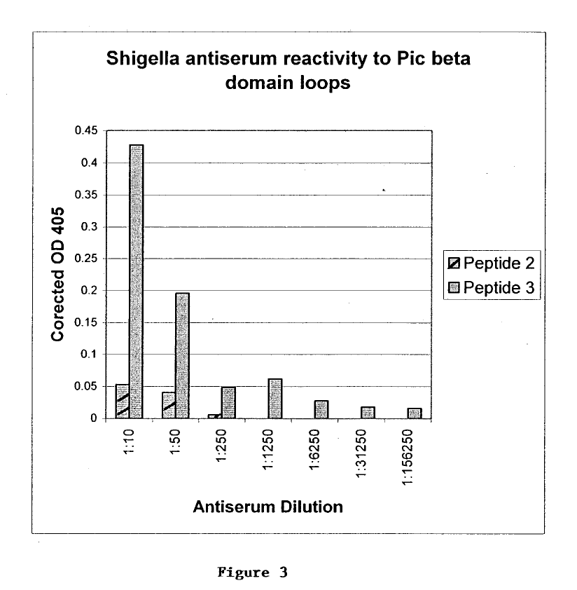

Through homology modeling of the secreted domain of pic, a beta-barrel model was produced. One view of the beta-barrel model illustrates a tube which forms a membrane pore arranged with intracellular domains. Another view indicates structural elements that are predicted to extend from the membrane surface and hence represent logical candidates for epitope selection. This analysis was used to further refine selection of structural epitopes for evaluation, which included selection of loop elements that link the beta-barrel structures and protrude out of the membrane. A list of peptide sequences comprising the external loop structures is presented in Table 5. To validate these loop structures as immunological targets, specific peptides were synthesized and screened against a polyclonal anti-Shigella flexneri antisera. Two peptides (peptide2, SEQ ID NO: 11; and peptide 3, SEQ ID NO:7) that were tested proved to have immune reactivity, hence confirming both the ability to replicate the loops in native configuration and their likely involvement in immune responses against Shigella (FIG. 3).

Confirmation of Diagnostic and Vaccine Utility of Autotransporter Domains

1. Computational Homology Modeling of the Shigella pic Gene, and Selection of Structural Domains as Candidate Epitopes

Computational homology modeling of the pic gene will be refined to include phylogenetic comparisons to other members of the autotransporter family. As indicated in Table 1, at least 15 members of this family have been isolated with primary gene sequence available. It is intended to generate a matrix comparing predicted structures for the beta-barrel domains of these proteins to the E. coli ompA gene used in the initial pic homology modeling. Phylogenetically conserved residues and structure for the chosen pic epitope loops will be identified, conserved structural homologies for exposed loop structures of the beta barrel will be ranked to evaluate these molecules as vaccine targets.

2. Design and Synthesis of Peptide Mimics of Chosen Structural Epitopes

Having chosen structural epitopes for analysis, peptide mimics will be synthesized in order to test immunogenicity and function. When excised from the native molecule and synthesized as a linear peptide, there is a low probability that the native structure will represent the dominant conformation, and hence a low probability that an immune response will generate antibodies interfering with the native molecule. Hence, computational modeling and design is a critical and novel tool for success.

Epitope Modeling

The ICM™ software suite will be used to computationally model the 4 identified epitope loops within a series of peptide scaffolds designed to conserve native structure. The approach will be to proceed as follows: 1) model and synthesize disulfide constrained peptide loops of estimated length 10 to 20 amino acid residues, modified with linker amino acid residues to reduce constraint and optimize structure; and 2) model and synthesize cyclic peptides of estimated 10 to 20 residue length, modified with linker amino acid residues, designed to reduce alternate conformations for the loop structure. In the above two cases, peptides will be synthesized for chemical conjugation to carrier molecules for immunogenicity studies.

The approach further includes defining the structural domains of the chosen epitopes, and designing linker residue additions necessary to embed the epitope in a molecular scaffold for presentation. The Hepatitis B core protein has been used as a vaccine delivery scaffold, based on it's properties of self assembly into a viral-like particle (VLP; (ref 33), such that the empty ghost particle can effectively stimulate a vigorous humoral immune response. The solved structure of the core protein, assembled into a VLP, allows insightful genetic insertion of target epitopes in order to “present” epitopes in a multiple array on the external surface of the VLP.

Epitope Synthesis

The next step is to prioritize early immunogenicity studies performed by conventional carrier protein adjuvant strategies to verify that structural epitopes can be replicated and that the resulting humoral response can block pic secretion function, hence validating target and epitope selection for a downstream vaccine approach. Chemically synthesized peptide epitopes have been screened for likelihood of native structure by reacting with antisera from patients exposed to Shigella. A further test would be to determine whether or not human antisera from volunteers infected with Shigella strains as part of past studies react against these molecules. While a positive result is encouraging, there are reasons a false negative result may be obtained. The primary reason would be low epitope titer within the sera, given the abundance of antigens presented during infection. It would be logical to affinity purify this antisera vs. the native pic protein, and perform relative binding affinity determinations.

Immunogenicity Testing of Structural Epitopes

Structural peptide epitopes will be synthesized through HPLC purification and analytical purification analysis. Peptide synthesis includes the attachment of biotin-modified amino acid residues, not involved in the structural epitope, to conjugate the epitopes to KLH as a carrier protein for immunization.

New Zealand white rabbits will be used for polyclonal sera production for each structural peptide. Immunizations will use incomplete Freund's adjuvant. Bleeds will be tested beginning at 1 month on bi-weekly schedules to determine antibody titer against peptide epitopes in an ELSA or dot blot format. Significant increases in titer relative to pre-bleeds merit sera collection and advanced testing. A failure to see significant increases in epitope titer, relative to KLH reactivity controls, will be interpreted to rule out the chemical synthesis strategy for that individual epitope.

The expression of the structural epitopes using VLP presentation will allow the use of the immunization strategies described above. Bleeds will be tested beginning at 1 month on bi-weekly schedules to determine antibody titer against purified VLP epitopes or VLP controls in an ELISA format. Significant increases in titer relative to pre-bleeds merit sera collection and advanced testing.

4. Functional Assay of Anti-Sera Raised Against Structural Epitopes for Interference with Protein Secretion Pathways

With the observation that significant titers against structural epitopes can be raised in rabbit immunogenicity studies, a next step will be to associate epitope specific antibodies for reactivity to native structure and functional interference with protein secretion. Antisera will be purified using peptide affinity chromatography, or in the case of VLP expressed epitopes, against immobilized VLP particles and subsequently depleted against a control VLP preparation lacking epitope. Relative dilutional titers will be determined.

Reactivity with Native Structure

Affinity purified polyclonal rabbit antisera for each pic epitope will be tested by in situ immunohistochemical staining of Shigella flexneri samples using standard techniques. A control strain, genetically deleted for the pic gene, is available. Reactivity against additional enteric organisms will be performed as well. It is possible that epitope cross-reactivity within Shigella, or between enteric organisms, could result in broader staining patterns than envisioned. Because of this, western blot experiments on membrane lysates from isolated bacterial strains will be performed. Inconclusive results will be followed by immunoprecipitation experiments using the affinity purified rabbit antisera, with confirmatory detection using antibodies reactive to the secreted domain of the pic protein.

Interference with Protein Secretion

The design of the long-term vaccine strategy is to block protein secretion by interfering with the pore structure utilized by these autotransporter proteins. Following demonstration of raising structural epitope specific antisera in immunogenicity studies, test of these sera for interference of pic secretion will proceed in two ways: production of mature pic protein, and blocking a mucin degradation property of pic.

Shigella flexneri will be incubated under standard laboratory conditions known to result in accumulation of pic in the cell supernatant. Standardized conditions for radioactively pulse-labeling pic will be supplemented with increasing titers of specific rabbit polyclonal sera to determine dilution endpoints at which pic secretion is blocked. Pic levels will be determined by relative quantification of sera for each epitope will be tested singly, and following dose determinations, used in combination in a matrix format. Control sera obtained from rabbit pre-bleeds will be used as a negative control.

The ability of the purified pic protein to cleave mucin can be measured using mucin purified from the cecum of mice and also using purified bovine submaxillary mucin. The pic protein is also able to cleave both gelatin and casein in zymogram gels. Shigella flexneri cultures will be reacted with test antisera, washed, and re-incubated in the presence of test antisera for the collection of supernatant. Supernatant will be tested relative to uninhibited control supernatant in the zymogram gel degradation assay.

This example describes structural epitopes of the Shigella pic gene that can be rationally designed and developed through immunogenicity testing. The ability to develop immune intervention and block protein secretion of the pic product will establish the methodology to block pathogenic effect using vaccine strategies targeting structural elements of protein secretion pathways.

### EXAMPLE 3

### Identification and Characterization of Yersinia Immunogenic Peptides

This example illustrates methods related to designing Yersinia pestis vaccines by computational molecular modeling of Yersinia toxin proteins and structural epitopes. The example includes the amino acid sequences which comprise selected epitopes within the Yersinia pestis murine toxin protein structure that are capable of inducing a protective immune response against the toxin or neutralizes the toxin activity in vitro and in vivo.

An immunogenic protein is constructed that presents the selected amino acid sequences as part of a synthetic HBc VLP structure that offers these epitopes apart from the other non-neutralizing antigenic determinants of the Y. pestis murine toxin, but held in a native conformational form and thereby capable of eliciting toxin-neutralizing antibodies. The structural epitopes include synthetic peptides either chemically conjugated to an assembled HBc VLP delivery vehicle or genetically inserted into HBc virus-like particles.

A structural homology model of Yersinia Murine Toxin (YMT) protein was designed using the ICM™ software suite (Molsoft LLC) using crystal structure of phospholipase D (PLD) from Streptomyces sp. as a template. Sequence homology between target sequence and template was low (˜21% identity; see, for example, Table 6; SEQ ID NOS:38 to 41); therefore, sequence-structure alignment was used, taking into account surface accessibility of aligned residues and alignment of predicted active site residues. Externally exposed polypeptide loops near the toxin active site were identified (see Table 6; SEQ ID NOS:12 to 28).

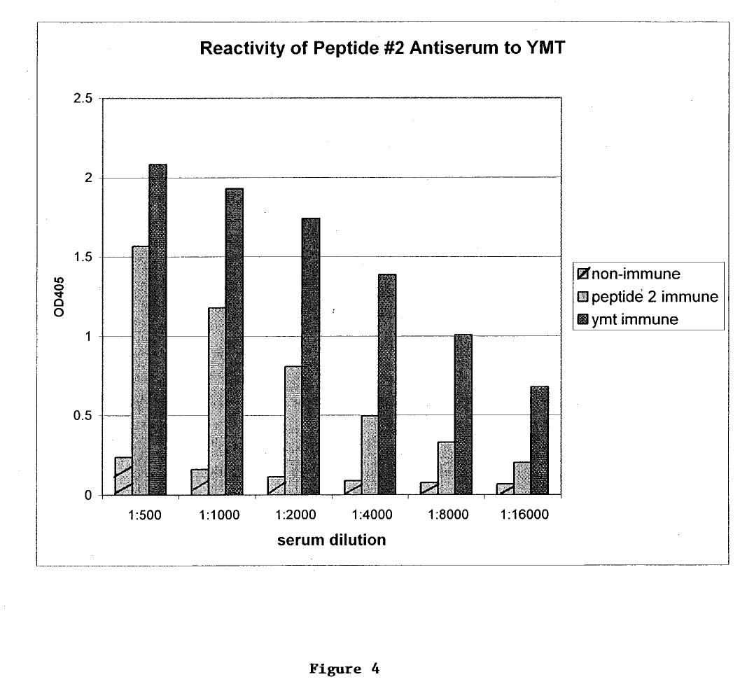

These selected loops were synthesized as multiple copies of 13 amino acid linear peptides on poly-lysine backbones to create multiple antigen peptides (MAP). New Zealand White rabbits were immunized subcutaneous (SC) with 500 μg of MAP peptide in Complete Freund's adjuvant, then boosted SC with 500 μg in Freund's incomplete at 2, 4, 6 and 8 weeks. Test bleeds showed high titers against peptide antigen (FIG. 4).

The targeted YMT structural epitopes were also genetically inserted into HBc VLPs and expressed in E. coli. Recombinant YMT-VLP proteins were affinity purified from bacterial cell lysates by a C-terminal polyhistidine tag. Rabbit antibodies raised against the YMT synthetic peptides recognized the target epitope as a structure on whole YMT protein, when tested by ELISA and western blot. These same antibodies also recognized the target epitope as presented on recombinant Hepatitis B core VLPs, in both ELISA and western blot assays.

TABLE 1

| Organism | Protein |
|---|---|
| Bordetella spp. | Pertactin, BrkA, TcfA, Vag8 |
| Dichelobacter nodosus | BprV, BprB, AprV2, BprX |
| Escherichia coli | EspP, Pet, Sat, Tsh, Pic AIDA-1, |
| | TibA, Ag43 |
| Haemophilus influenzae | lgAl protease, Hap, Hia, Hsf |
| Helicobacter mustelae | Hsr |
| Helicobacter pylori | VacA, BabA |
| Moraxella catarrhalis | UspA1, UspA2, UspA2h |
| Neisseria spp. | lgA1 protease |
| Paste urella haemolytica | Ssal |
| Pseudomonas aeruginosa | EstA |
| Pseudomonas fluorescens | PspA, PspB |
| Rickettsiales | rOmpA, rOmpB |
| Salmonella enterica | ApeE |
| serovar Typhimunium | |
| Serratia marcesens | PrtS, PrT, Ssp-HI, Ssp-HZ |
| Shigella flexneri | SepA, Pic, SigA, IcsA |
| Xenorhabdus luminescens | PlaA |

TABLE 2
Functional and immunogenic features of
PA Domain 4(SEQ ID NO:30)

| Residues | Feature | Comments |
|---|---|---|
| 596-735 | Full C-terminal Domain 4 | ATR-binding domain |
| | | Stand alone protective antigen |
| 679-693 | “Small loop”, ATR binding | Identified by mutation analysis |
| | determinant | |
| 704-722 | “Large loop”, indirectly | Mutation analysis, truncation of |
| | involved in ATR binding | C-terminus |
| 671-721 | mAb binding region, | mAbs 3B6, 14B7 and 10E10 |
| | both loops | inhibit binding to ATR |

TABLE 3
Relative stability of PA subfolds

| | | | | Surface | Distance between |
|---|---|---|---|---|---|
| Domain | N-term | C-term | Energy Strain | energy | termini |
| PA-4 | 606 | 705 | 0.416 | −0.071 | 8.86 |
| PA-4 | 606 | 735 | 0.419 | −0.069 | 30.9 |
| PA-4 | 606 | 706 | 0.421 | −0.070 | 9.6 |
| PA-4 | 606 | 704 | 0.422 | −0.070 | 5.0 |
| PA-4 | 606 | 734 | 0.434 | −0.068 | 33.1 |
| PA-4 | 607 | 703 | 0.446 | −0.070 | 5.6 |
| PA-4 | 606 | 732 | 0.451 | −0.067 | 33.7 |
| PA-4 | 604 | 707 | 0.454 | −0.069 | 11.1 |
| PA-4 | 606 | 730 | 0.455 | −0.067 | 28.1 |
| PA-4 | 606 | 733 | 0.460 | −0.067 | 36.5 |
| PA-1 | 17 | 153 | 1.296 | −0.074 | 5.8 |
| PA-2 | 261 | 454 | 1.830 | −0.055 | 9.0 |
| PA-3 | 487 | 594 | 0.778 | −0.063 | 29.9 |

TABLE 4
Prediction of chimeric PA-VLP stability. Variation is calculated
from four different independent runs.

| | | Energy Strain in | |
|---|---|---|---|
| Linker1 | Linker2 | monomer | Energy in 23-mer |
| (G) 3* | (G) 4** | 0.38± 0.03 | 100.9± 2.7 |
| (G) 4 | (G) 5 | 0.36± 0.05 | 104.6± 3.5 |
| (G) 5 | (G) 6 | 0.36± 0.03 | 105.7± 1.7 |
| (G) 2 | (G) 3 | 0.39± 0.10 | 106.0± 2. |
| GSAGS | GSAGSA | 0.46± 0.04 | 108.2± 4.3 |
| (G) 3 | (G) 3 | 0.40± 0.08 | 109.6± 1.8 |
| (G) 5 | (G) 5 | 0.45± 0.05 | 112.7± 2.4 |
| GSAGSA | GSAGSAG | 0.43± 0.07 | 112.7± 3.7 |
| (G) 4 | (G) 4 | 0.42± 0.05 | 114.7± 1.5 |
| (G) 6 | (G) 6 | 0.40± 0.09 | 119.1± 1.8 |

*Single letter amino acid code.
**SEQ ID NOS:, as follows: G (4), SEQ ID NO: 37; G (5), SEQ ID NO: 42; G (6), SEQ ID NO: 43; GSAGS, SEQ ID NO: 44; GSAGSA, SEQ ID NO: 45; GSAGSAG, SEQ ID NO: 46.

TABLE 5

| | | | |
|---|---|---|---|
| P/E | Residue # | Length | Sequence |

Best (predicted by all four methods)

| | | | |
|---|---|---|---|
| P | 1144:1149 | 6 | ELDGVD (1)* |
| E | 1162:1174 | 13 | SADSHAFSGKTKS (2) |
| P | 1341:3343 | 3 | KDN (3) |
| E | 1354:1362 | 9 | AFGKYNVDN (4) |

Good (predicted by three methods)

| | | | |
|---|---|---|---|
| E | 1119:1133 | 15 | SGAGSADGGYSDNYT |
| | | | (5) |
| P | 1186:1188 | 3 | ESG (6) |
| E | 1206:1216 | 11 | GNFASLGTKHY 7) |
| P | 1231:1236 | 6 | YHLTED 8) |
| E | 1252:1261 | 10 | GKTFRWKDGD (9) |
| P | 1305:1309 | 5 | LLNNG (10) |
| E | 1316:1328 | 13 | ASGBKRIKGEKDS (11) |

*Number in parentheses is SEQ ID NO:

Table 6.
Regions of homology between Ymt and hPLD1:

| | | | | |
|---|---|---|---|---|
| Ymt | 132 | NHTKI MASDGTEALVGGHNMNMDLF | 156 | (38)* |
| hLD1 | 463 | NHEKLVII DQSVAFBGGI NLAYGRW | 487 | (39) |
| Ymt | 467 | GNHAKLMI I DDELYVVGSDNLYPGYL | 492 | (40) |
| hLD1 | 894 | YVHSKLLI ADDNTVII GSANI NDRSM | 919 | (41) |

Ymt surface exposed loops:

| | | proximity to |
|---|---|---|
| residue # | sequence | active sight |
| 1:13(12)* | MTEVLRNSRYRCD | − |
| 36:48(13) | LSKKMGRTQPTQY | − |
| 52:64(14) | FGQSPTVFMNGLS | + |
| 68:80(15) | NGSPDFVAFKSEL | − |
| 76:88(16) | FKSELIQLIKERG | +/− |
| 102:114(17) | FFRIEEGLATSFM | − |
| 151:163(18) | MNMDLFRNYPPVH | + |
| 188:200(19) | NSDLLKKEYFDYE | − |
| 211:223(20) | YDKPEDPLKSSVA | − |
| 243:255(21) | QKVATRISEYENM | − |
| 288:300(22) | KEQLIKNARRIIR | +/− |
| 384:396(23) | DGSRADALKRI LI | + |
| 389:401(24) | DALKRI LI APFFF | +/− |
| 410:422(25) | TIEGETYKWPDLE | − |
| 433:455(26) | GII GSALMSAIKG | − |
| 502:514(27) | GKDAVNELMKSYW | − |
| 506:518(28) | VNELMKSYWEPLW | − |

* Number in parentheses is SEQ ID NO:

REFERENCES CITED: Each of the following articles is incorporated herein by reference.

1. Abagyan, R. & Totrov, M. Biased probability Monte Carlo conformational searches and electrostatic calculations for peptides and proteins. J Mol Biol 235, 983-1002. (1994).

2. Totrov, M. & Abagyan, R. Rapid boundary element solvation electrostatics calculations in folding simulations: successful folding of a 23-residue peptide. Biopolymers 60, 124-33 (2001).

3. Totrov, M. & Abagyan, R. The contour-buildup algorithm to calculate the analytical molecular surface. J Struct Biol 116, 138-43. (1996).

4. Borchert, T. V., Abagyan, R., Jaenicke, R. & Wierenga, R. K. Design, creation, and characterization of a stable, monomeric triosephosphate isomerase. Proc Natl Acad Sci USA 91, 1515-8. (1994).

5. Norledge, B. V. et al. Modeling, mutagenesis, and structural studies on the fully conserved phosphate-binding loop (loop 8) of triosephosphate isomerase: toward a new substrate specificity. Proteins 42, 383-9. (2001).

6. Thanki, N. et al. Protein engineering with monomeric triosephosphate isomerase (monoTIM): the modelling and structure verification of a seven-residue loop. Protein Eng 10, 159-67. (1997).

7. Borchert, T. V. et al. Three new crystal structures of point mutation variants of monoTIM: conformational flexibility of loop-1, loop-4 and loop-8. Structure 3, 669-79. (1995).

8. Abagyan, R. et al. Homology modeling with internal coordinate mechanics: deformation zone mapping and improvements of models via conformational search. Proteins Suppl, 29-37. (1997).

9. Cardozo, T., Totrov, M. & Abagyan, R. Homology modeling by the ICM method. Proteins 23, 403-14. (1995).

10. Welkos, S., Little, S., Friedlander, A., Fritz, D. & Fellows, P. The role of antibodies to Bacillus anthracis and anthrax toxin components in inhibiting the early stages of infection by anthrax spores. Microbiology 147, 1677-85. (2001).

11. Fowler, K., McBride, B. W., Turnbull, P. C. & Baillie, L. W. Immune correlates of protection against anthrax. J Appl Microbiol 87, 305. (1999).

12. Pitt, M. L. et al. In vitro correlate of immunity in an animal model of inhalational anthrax. J Appl Microbiol 87, 304. (1999).

13. Reuveny, S. et al. Search for correlates of protective immunity conferred by anthrax vaccine. Infect Immun 69, 2888-93. (2001).

14. Ivins, B. E. et al. Comparative efficacy of experimental anthrax vaccine candidates against inhalation anthrax in rhesus macaques. Vaccine 16, 1141-8. (1998).

15. Little, S. F., Ivins, B. E., Fellows, P. F. & Friedlander, A. M. Passive protection by polyclonal antibodies against Bacillus anthracis infection in guinea pigs. Infect Immun 65, 5171-5. (1997).

16. Sterne, M. Avirulent anthrax vaccine. Onderstepoort J. Vet. Sci. Animal Ind. 21, 41-43 (1937).

17. Ivins, B. E., Welkos, S. L., Knudson, G. B. & Little, S. F. Immunization against anthrax with aromatic compound-dependent (Aro-) mutants of Bacillus anthracis and with recombinant strains of Bacillus subtilis that produce anthrax protective antigen. Infect Immun 58, 303-8. (1990).

18. Little, S. F. & Knudson, G. B. Comparative efficacy of Bacillus anthracis live spore vaccine and protective antigen vaccine against anthrax in the guinea pig. Infect Immun 52, 509-12. (1986).

19. Pezard, C., Weber, M., Sirard, J. C., Berche, P. & Mock, M. Protective immunity induced by Bacillus anthracis toxin-deficient strains. Infect Immun 63, 1369-72. (1995).

20. Stepanov, A. V., Marinin, L. I., Pomerantsev, A. P. & Staritsin, N. A. Development of novel vaccines against anthrax in man. J Biotechnol 44, 155-60. (1996).

21. Price, B. M. et al. Protection against anthrax lethal toxin challenge by genetic immunization with a plasmid encoding the lethal factor protein. Infect Immun 69, 4509-15. (2001).

22. Brossier, F., Weber-Levy, M., Mock, M. & Sirard, J. C. Protective antigen-mediated antibody response against a heterologous protein produced in vivo by Bacillus anthracis. Infect Immun 68, 5731-4. (2000).

23. Kobiler, D. et al. Efficiency of protection of guinea pigs against infection with Bacillus anthracis spores by passive immunization. Infect Immun 70, 544-60. (2002).

24. Schaffer, A. A. et al. Improving the accuracy of PSI-BLAST protein database searches with composition-based statistics and other refinements. Nucleic Acids Res 29, 2994-3005. (2001).

25. Cardozo, T., Batalov, S. & Abagyan, R. Estimating local backbone structural deviation in homology models. Comput Chem 24, 13-31. (2000).

26. Abagyan, R. A. & Batalov, S. Do aligned sequences share the same fold? J Mol Biol 273, 355-68. (1997).

27. Maiorov, V. & Abagyan, R. Energy strain in three-dimensional protein structures. Fold Des 3, 259-69 (1998).

28. Gaur, R., Gupta, P. K., Banerjea, A. C. & Singh, Y. Effect of nasal immunization with protective antigen of Bacillus anthracis on protective immune response against anthrax toxin. Vaccine 20, 2836-9. (2002).

29. Flick-Smith, H. C. et al. Mucosal or parenteral administration of microsphere-associated Bacillus anthracis protective antigen protects against anthrax infection in mice. Infect Immun 70, 2022-8. (2002).

30. Flick-Smith, H. C. et al. A recombinant carboxy-terminal domain of the protective antigen of Bacillus anthracis protects mice against anthrax infection. Infect Immun 70, 1653-6. (2002).

31. Shlyakhov, E. N. & Rubinstein, E. Human live anthrax vaccine in the former USSR. Vaccine 12, 727-30. (1994).

32. Use of anthrax vaccine in the United States: recommendations of the Advisory Committee on Immunization Practices. J Toxicol Clin Toxicol 39, 85-100 (2001).

33. Pumpens, P. & Grens, E. HBV core particles as a carrier for B cell/T cell epitopes. Intervirology 44, 98-114 (2001).

34. Fouts, T. R. et al. Expression and characterization of a single-chain polypeptide analogue of the human immunodeficiency virus type 1 gp120-CD4 receptor complex. J Virol 74, 11427-36. (2000).

35. Maiorov, V. & Abagyan, R. A new method for modeling large-scale rearrangements of protein domains. Proteins 27, 410-24. (1997).

36. Pannifer, A. D. et al. Crystal structure of the anthrax lethal factor. Nature 414, 229-33. (2001).

37. Barlow, D. J., Edwards, M. S. & Thornton, J. M. Continuous and discontinuous protein antigenic determinants. Nature 322, 747-8. (1986).

38. Thornton, J. M., Edwards, M. S., Taylor, W. R. & Barlow, D. J. Location of ‘continuous’ antigenic determinants in the protruding regions of proteins. Embo J 5, 409-13. (1986).

39. Connolly, M. L. The molecular surface package. J Mol Graph 11, 139-41 (1993).

40. Little, S. F., Leppla, S. H. & Friedlander, A. M. Production and characterization of monoclonal antibodies against the lethal factor component of Bacillus anthracis lethal toxin. Infect Immun 58, 1606-13. (1990).

41. Little, S. F. et al. Characterization of lethal factor binding and cell receptor binding domains of protective antigen of Bacillus anthracis using monoclonal antibodies. Microbiology 142, 707-15. (1996).

42. Price, L. B., Hugh-Jones, M., Jackson, P. J. & Keim, P. Genetic diversity in the protective antigen gene of Bacillus anthracis. J Bacteriol 181, 2358-62. (1999).

43. Borisova, G. et al. Spatial structure and insertion capacity of immunodominant region of hepatitis B core antigen. Intervirology 39, 16-22 (1996).

44. Little, S. F., Leppla, S. H. & Cora, E. Production and characterization of monoclonal antibodies to the protective antigen component of Bacillus anthracis toxin. Infect Immun 56, 1807-13. (1988).A

Although the invention has been described with reference to the above examples, it will be understood that modifications and variations are encompassed within the spirit and scope of the invention. Accordingly, the invention is limited only by the following claims.

What is claimed is:

1. A method for identifying an immunogenic peptide representative of a structural element of a target protein having a known three dimensional structure, comprising:
selecting epitopes of the target protein based on the three dimensional structure of the target protein, thereby obtaining selected epitopes;
detecting, in a molecular model of a polypeptide comprising a selected epitope linked to a scaffold protein, an epitope having a three dimensional conformation corresponding to the three dimensional structure of the epitope in the target protein, thereby identifying a candidate immunogenic peptide representative of a structural element of the target protein; and
detecting that antibodies induced by the candidate immunogenic peptide selectively bind to the target protein, thereby identifying an immunogenic peptide representative of a structural element of the target protein.

2. The method of claim 1, wherein the scaffold protein comprises a viral coat protein.

3. The method of claim 2, wherein the viral coat protein comprises a Hepatitis B core protein.

4. The method of claim 1, wherein the selected epitope is linked to the scaffold protein via a linker moiety.

5. The method of claim 4, wherein the linker moiety comprises a peptide.

6. The method of claim 1, wherein the polypeptide comprising a selected epitope linked to a scaffold protein is a fusion protein.

7. The method of claim 1, wherein the scaffold protein comprises a poly(amino acid).

8. The method of claim 7, wherein the poly(amino acid) comprises poly(lysine).

9. The method of claim 1, wherein the target protein comprises a protein of an infectious microorganism.

10. The method of claim 9, wherein the infectious microorganism is a bacterium.

11. The method of claim 10, wherein the infectious microorganism causes anthrax.

12. The method of claim 11, wherein the target protein comprises anthrax protective antigen.

13. The method of claim 11, wherein the target protein comprises anthrax lethal factor.

14. The method of claim 2, further comprising assembling a plurality of immunogenic peptides, each immunogenic peptide comprising an epitope linked to a viral coat protein, into a virus-like particle.

15. The method of claim 14, wherein epitopes of the immunogenic peptides of the plurality are the same.

16. The method of claim 14, wherein epitopes of the immunogenic peptides of the plurality comprise anthrax protective antigen epitopes and anthrax lethal factor epitopes.

17. An immunogenic peptide identified according to the method of claim 1.

18. An isolated peptide, consisting of:
amino acid residues 606 to 705 of SEQ ID NO:30;
amino acid residues 606 to 735 of SEQ ID NO:30;
amino acid residues 606 to 706 of SEQ ID NO:30;
amino acid residues 606 to 704 of SEQ ID NO:30;
amino acid residues 606 to 734 of SEQ ID NO:30;
amino acid residues 607 to 703 of SEQ ID NO:30;
amino acid residues 606 to 732 of SEQ ID NO:30;
amino acid residues 604 to 707 of SEQ ID NO:30;
amino acid residues 606 to 730 of SEQ ID NO:30; or
amino acid residues 606 to 733 of SEQ ID NO:30.

19. An isolated antibody that selectively binds a peptide of claim 18, provided said antibody does not substantially bind a peptide comprising:
amino acid residues 596 to 735 of SEQ ID NO:30;
amino acid residues 679 to 693 of SEQ ID NO:30;
amino acid residues 703 to 722 of SEQ ID NO:30; or
amino acid residues 671 to 721 of SEQ ID NO:30.

20. An isolated polynucleotide encoding the peptide of claim 18.

21. An isolated peptide, consisting of:
amino acid residues 17 to 153 of SEQ ID NO:30;
amino acid residues 261 to 454 of SEQ ID NO:30; or
amino acid residues 487 to 594 of SEQ ID NO:30.

22. A composition, comprising at least a first peptide of claim 18 operatively linked to at least a first heterologous molecule.

23. The composition of claim 22, wherein the first heterologous molecule comprises a linker moiety.

24. The composition of claim 23, wherein the linker moiety comprises a peptide linker.

25. The composition of claim 24, wherein the peptide linker comprises an oligo(glycine) linker.

26. The composition of claim 24, wherein the peptide linker comprises a glycine-serine-alanine linker.

27. The composition of claim 22, wherein the first heterologous molecule comprises a heterologous peptide.

28. The composition of claim 27, which comprises a fusion protein.

29. The composition of claim 27, wherein the heterologous peptide comprises a scaffold protein.

30. The composition of claim 29, wherein the scaffold protein comprises a viral coat protein.

31. The composition of claim 30, wherein the viral coat protein comprises a Hepatitis B core protein.

32. The composition of claim 31, wherein the Hepatitis B core protein consists of amino acid residues 1 to 77 and amino acid residues 80 to 149 of SEQ ID NO:34, and wherein the peptide of claim B 1 is operatively linked to amino acid residue 77 and to amino acid residue 80 of SEQ ID NO:34.

33. The composition of claim 32, wherein the peptide of claim 18 is operatively linked to amino acid residue 77 via a first linker moiety and is operatively linked to amino acid residue 80 via a second linker moiety.

34. The method of claim 33, wherein the first linker moiety and the second linker moiety are the same.

35. The method of claim 33, wherein each of the first linker moiety and the second linker moiety is a peptide.

36. The composition of claim 22, further comprising at least a second heterologous molecule.

37. The composition of claim 36, wherein at least the first heterologous molecule comprises a linker moiety or a scaffold protein.

38. The composition of claim 37, wherein the first heterologous molecule comprises a linker moiety and the second heterologous molecule comprises a scaffold protein, and wherein the peptide of claim 18 is operatively linked to the scaffold protein via the linker moiety.

39. The composition of claim 36, comprising, in operative linkage, a first portion of a scaffold protein, a first peptide linker, the peptide of claim 18, a second peptide linker, and a second portion of the scaffold protein.

40. The composition of claim 39, wherein the scaffold protein comprises a viral coat protein.

41. The composition of claim 39, which comprises an amino acid sequence as set forth in SEQ ID NO:36.

42. The composition of claim 40, which comprises a plurality of polypeptides, wherein polypeptides of the plurality comprise a peptide of claim B 1 operatively linked to a viral coat protein.

43. The composition of claim 42, wherein polypeptides of the plurality are assembled to form a virus-like particle.

44. The composition of claim 22, wherein the first heterologous molecule comprises a tag.

45. The composition of claim 44, wherein the tag comprises a peptide tag.

46. The composition of claim 45, wherein the peptide tag comprises an oligo(histidine) tag.

47. The composition of claim 22, which is comprises immunogenic peptide representative of a structural element of the target protein.

48. The composition of claim 47, further comprising a carrier.

49. The composition of claim 48, wherein carrier comprises an adjuvant.

50. A polynucleotide encoding the composition of claim 22.

51. The polynucleotide of claim 50, which comprises SEQ ID NO:35.

52. The polynucleotide of claim 50, which is contained in a vector.

53. The polynucleotide of claim 52, wherein the vector is a viral vector.

54. The polynucleotide of claim 50, which is contained in a matrix.

55. The polynucleotide of claim 54, wherein the matrix comprises liposomes or microbubbles.

56. A method of stimulating an immune response in a subject, comprising administering the composition of claim 22, or a polynucleotide encoding the composition, to a subject under conditions suitable for stimulating an immune response.

57. The method of claim 56, wherein the immune response comprises a protective immune response.

58. The method of claim 56, wherein the subject is a mammalian subject.

59. The method of claim 56, wherein the subject is a human subject.

60. A method for identifying an immunogenic peptide representative of a structural element of a target protein having a known amino acid sequence but an unknown three dimensional structure, comprising:
generating a molecular model of a three dimensional structure of the target protein based on the three dimensional structure of a homologous protein;
selecting epitopes of the target protein based on the molecular model of the three dimensional structure of the target protein, thereby obtaining selected epitopes;
detecting, in a molecular model of a chimeric polypeptide comprising at least one selected epitope having a constrained structure, an epitope having a three dimensional conformation corresponding to the molecular model of the three dimensional structure of the epitope in the target protein, thereby identifying a candidate immunogenic peptide representative of a structural element of the target protein; and
detecting that antibodies induced by the candidate immunogenic peptide selectively bind to the target protein, thereby identifying an immunogenic peptide representative of a structural element of the target protein.

61. The method of claim 60, wherein the chimeric polypeptide comprises a plurality of linked selected epitopes.

62. The method of claim 61, wherein the selected epitopes of the plurality are the same.

63. The method of claim 60, wherein the chimeric polypeptide further comprises at least one linker peptide, which is linked to at least one selected epitope of the chimeric polypeptide.

64. The method of claim 60, wherein the constrained structure of the selected epitope is due to at least one disulfide bond in the chimeric polypeptide.

65. The method of claim 60, wherein the constrained structure of the selected epitope is due to cyclization of the chimeric polypeptide.

66. The method of claim 60, wherein the chimeric polypeptide further comprises a scaffold protein.

67. The method of claim 66, wherein the scaffold protein comprises a viral coat protein.

68. The method of claim 67, wherein the viral coat protein comprises a Hepatitis B core protein.

69. The method of claim 66, wherein the scaffold protein comprises a poly(amino acid).

70. The method of claim 69, wherein the poly(amino acid) comprises poly(lysine).

71. The method of claim 66, wherein the selected epitope is linked to the scaffold protein via a linker moiety.

72. The method of claim 71, wherein the linker moiety comprises a peptide.

73. The method of claim 60, wherein the target protein comprises a protein of an infectious microorganism.

74. The method of claim 73, wherein the infectious microorganism is a bacterium.

75. The method of claim 74, wherein the target protein comprises an autotransporter.

76. The method of claim 75, wherein the autotransporter comprises an autotransporter as set forth in Table 1.

77. The method of claim 74, wherein the infectious microorganism is a Shigella species.

78. The method of claim 77, wherein the Shigella species is Shigella flexneri.

79. The method of claim 77, wherein the target protein comprises a Shigella protease involved in colonization (pic) gene product.

80. The method of claim 74, wherein the infectious microorganism is a Yersinia species.

81. The method of claim 80, wherein the Yersinia species is Yersinia pestis.

82. The method of claim 80, wherein the target protein comprises a Yersinia murine toxin (YMt).

83. The method of claim 67, further comprising assembling a plurality of immunogenic peptides, each immunogenic peptide comprising an epitope linked to a viral coat protein, into a virus-like particle.

84. The method of claim 83, wherein epitopes of the immunogenic peptides of the plurality are the same.

85. An immunogenic peptide identified according to the method of claim 60.

86. An isolated peptide, consisting of a peptide as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1 1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, or SEQ ID NO:28.

87. An isolated polynucleotide encoding the peptide of claim 86.

88. The polynucleotide of claim 87, which is contained in a vector.

89. The polynucleotide of claim 88, wherein the vector is a viral vector.

90. The polynucleotide of claim 87, which is contained in a matrix.

91. The polynucleotide of claim 90, wherein the matrix comprises liposomes or microbubbles.

92. A composition, comprising at least a first peptide of claim 86 operatively linked to at least a first heterologous molecule.

93. The composition of claim 92, comprising a plurality of peptides of claim 86, wherein the peptides of the plurality are linked to each other.

94. The composition of claim 93, wherein peptides of the plurality are the same.

95. The composition of claim 92, wherein the first heterologous molecule comprises a linker moiety.

96. The composition of claim 95, wherein the linker moiety comprises a peptide linker.

97. The composition of claim 92, wherein the first heterologous molecule comprises a first heterologous peptide.

98. The composition of claim 97, wherein the first heterologous peptide comprises a scaffold protein.

99. The composition of claim 98, wherein the scaffold protein comprises a poly(amino acid).

100. The composition of claim 99, wherein the poly(amino acid) comprises poly(lysine).

101. The composition of claim 98, wherein the scaffold protein comprises a viral coat protein.

102. The composition of claim 101, wherein the viral coat protein comprises a Hepatitis B core protein.

103. The composition of claim 102, wherein the Hepatitis B core protein consists of amino acid residues 1 to 77 and amino acid residues 80 to 149 of SEQ ID NO:34, and wherein the peptide of claim 86 is operatively linked to amino acid residue 77 and to amino acid 80 of SEQ ID NO:34.

104. The composition of claim 101, comprising a plurality of peptides of claim 86, wherein each peptide of the plurality is operatively linked to a viral coat protein.

105. The composition of claim 104, which are assembled to form a virus-like particle.

106. The composition of claim 92, further comprising at least a second heterologous molecule.

107. The composition of claim 106, wherein at least the first heterologous molecule comprises a linker moiety or a scaffold protein.

108. The composition of claim 106, wherein the first heterologous molecule comprises a linker moiety and the second heterologous molecule comprises a scaffold protein, and wherein the linker moiety operatively links the peptide of claim NI to the scaffold protein.

109. The composition of claim 106, comprising, in operative linkage, a first portion of a scaffold protein, a first peptide linker, the peptide of claim N1, a second peptide linker, and a second portion of the scaffold protein.

110. The composition of claim 109, wherein the scaffold protein comprises a viral coat protein.

111. The composition of claim 92, wherein the first heterologous molecule comprises a tag.

112. The composition of claim 92, wherein the first heterologous molecule comprises a carrier protein.

113. The composition of claim 92, which is an immunogenic composition.

114. The composition of claim 113, further comprising an adjuvant.

115. A method of stimulating an immune response in a subject, comprising administering the composition of claim 92, or a polynucleotide encoding the composition, to a subject under conditions suitable for stimulating an immune response.

116. The method of claim 115, wherein the immune response comprises a protective immune response.

117. The method of claim 115, wherein the subject is a mammalian subject.

118. The method of claim 115, wherein the subject is a human subject.
